(19) United States
(12) Patent Application Publication
(10) Pub. No.: US 2010/0184094 A1
(43) Pub. Date: Jul. 22, 2010

(54) USE OF MDCK CELLS IN THE EVALUATION OF CHOLESTEROL MODULATORS

(76) Inventors: Maria L. Garcia, Edison, NJ (US); Martin G. Kohler, Scotch Plains, NJ (US); Adam Weinglass, East Brunswick, NJ (US)

Correspondence Address: MERCK, P O BOX 2000, RAHWAY, NJ, 07065-0907, US

(21) Appl. No.: 12/665,110

(22) PCT Filed: Jun. 25, 2008
(86) PCT No.: PCT/US08/68121

§ 371 Date: Dec. 17, 2009

Related U.S. Application Data
(60) Provisional application No. 60/937,798, filed on Jun. 28, 2007.

(51) Int. Cl. G01N 33/53; C07K 14/00; C07H 21/00; C12N 15/74; C12N 5/071

(52) U.S. Cl. 435/7.21; 530/350; 536/23.1; 435/320.1; 435/350

(57) ABSTRACT

A novel use for MDCK cells in the evaluation of cholesterol modulators is provided. In particular, methods for detecting substances which bind to NPC1L1 and block intestinal cholesterol absorption are provided. Such substances are of use in the treatment of individuals with hypercholesterolemia. The various assays may additionally be employed for studying NPC1L1 function.

### CROSS-REFERENCE TO RELATED APPLICATIONS

This application claims the benefit of U.S. Provisional Application No. 60/937,798 filed on Jun. 28, 2007.

### FIELD OF THE INVENTION

The present invention relates to a novel use of an existing cell line for the identification and study of cholesterol modulators.

### BACKGROUND OF THE INVENTION

A factor leading to the development of vascular disease, a leading cause of death in industrialized nations, is elevated serum cholesterol. It is estimated that 19% of Americans between 20 and 74 years of age have high serum cholesterol. The most prevalent form of vascular disease is arteriosclerosis, a condition associated with the thickening and hardening of the arterial wall. Arteriosclerosis of the large vessels is referred to as atherosclerosis. Atherosclerosis is the predominant underlying factor in vascular disorders such as coronary artery disease, aortic aneurysm, arterial disease of the lower extremities and cerebrovascular disease. Adequate regulation of serum cholesterol is, therefore, of critical import for the prevention and treatment of vascular disease.

Whole-body cholesterol homeostasis in mammals and animals involves the regulation of various pathways including intestinal cholesterol absorption, cellular cholesterol trafficking, dietary cholesterol and modulation of cholesterol biosynthesis, bile acid biosynthesis, steroid biosynthesis and the catabolism of the cholesterol-containing plasma lipoproteins.

The effective identification and study of critical factors involved in cholesterol homeostasis through such pathways relies significantly on the availability of appropriate cell lines that express and model the critical proteins and many cellular factors that contribute to such processes.

Niemann-Pick C1-Like 1 (“NPC1L1”) protein is one such critical component of cholesterol uptake in enterocytes. NPC1L1 is an N-glycosylated protein comprising a YQRL (SEQ ID NO: 1) motif (i.e., a trans-golgi network to plasma membrane transport signal; see Bos et al., 1993 EMBO J. 12:2219-2228; Humphrey et al., 1993 J. Cell. Biol. 120:1123-1135; Ponnambalam et al., 1994 J. Cell. Biol. 125:253-268; and Rothman et al., 1996 Science 272:227-234). NPC1L1 exhibits limited tissue distribution and gastrointestinal abundance. While the role of NPC1L1 is not well defined (Huff et al., 2006 Arterioscler, Thromb, Vase. Biol. 26:2433-2438), administration of compounds that target NPC1L1 block cholesterol absorption and are effective in the treatment of hypercholesterolemia. Accordingly, the further study of the underlying mechanism of NPC1L1 is of significant import. Obtaining a full understanding of the molecular mechanism of NPC1L1, like other critical components involved in cholesterol homeostasis, however, requires identification of an appropriate in vitro system for detailed biochemical studies. Enterocytes, while the current cell line of choice, have proven difficult to culture in vitro; Simon-Assmann et al., 2007 Cell. Biol. Toxicol. 23:241-256. Several groups have expressed NPC1L1 in recombinant systems (Iyer et al., 2005 Biochim. Biophys. Acta 1722:282-292; Davies et al., 2005 J. Biol. Chem. 280:12710-12720; Yu et al., 2006 J. Biol. Chem. 281:6616-6624) or, in the alternative, identified cell lines, such as CaCo-2 cells (Davies et al., 2005J. Biol. Chem. 280:12710-12720, During et al., 2005 J. Nutr. 135:2305-2312; Sane et al., 2006 J. Lipid Res. 47(10:2112-2120) and HepG2 cells (Davies et al., 2005 J Biol. Chem. 280:12710-12720; Yu et al., 2006 J. Biol. Chem. 281:6616-6624) that endogenously express NPC1L1. While these strategies have seemingly presented a path forward, their utility is somewhat limited. They are either not fully representative of the natural environment, responsible proteins and systems (recombinant systems) or they exhibit discrepancies in the sub-cellular localization and functionality of expressed NPC1L1 (CaCo-2 and HepG2 cells). Said shortcomings ultimately raise the question of whether they are appropriate surrogates for studying the mechanism of NPC1L1.

Development of an appropriate in vitro system is critical to enable the study of not just NPC1L1 but all critical cellular components involved in cholesterol absorption.

The present invention addresses this need by providing a novel system for using an existing cell line which expresses and models such critical components and pertinent cellular factors.

### SUMMARY OF THE INVENTION

The present invention relates to a novel method for using polarized Madin-Darby Canine Kidney (“MDCK”) cells in the study and identification of cholesterol modulators (i.e., compounds, biologicals and other molecules that impact cholesterol homeostasis through an effect on cholesterol absorption, transport, synthesis and/or catabolism). In additional embodiments, the present invention relates to the use of MDCK cells for use in the identification and study of cellular proteins or factors involved in the regulation of cholesterol homeostasis.

In specific embodiments, the method comprises contacting MDCK cells with a candidate NPC1L1 modulator and identifying those candidate NPC1L1 modulators that bind to NPC1L1. Such experiments may be performed along with a control experiment wherein NPC1L1-dependent binding is minimal or absent, including but not limited to a different cell line not expressing NPC1L1, cells from which genomic NPC1L1 DNA has been disrupted or deleted, or cells where endogenous NPC1L1 RNA has been depleted, for example, by RNAi.

In specific embodiments, the present invention relates to a method which comprises contacting the MDCK cells with a detectably labeled known or previously characterized NPC1L1 modulator, and a candidate NPC1L1 modulator, and determining whether the candidate modulator binds to NPC1L1, displacing the detectably labeled NPC1L1 modulator, essentially competing for binding with the known NPC1L1 modulator. In such instances where the candidate NPC1L1 modulator competes with the known NPC1L1 modulator, the candidate NPC1L1 modulator binds NPC1L1 selectively and is a likely inhibitor of sterol (e.g., cholesterol) and 5α-stanol absorption.

The present invention also relates to methods for identifying NPC1L1 modulators which comprises: (a) saturating NPC1L1 binding sites on MDCK cells with a detectably labeled previously characterized NPC1L1 modulator, (b) measuring the amount of bound label, (c) contacting the cells with an unlabeled candidate NPC1L1 modulator (or, in the alternative, a candidate modulator bearing a distinct label); and (d) measuring the amount of bound label remaining; displacement of the label indicating the presence of an NPC1L1 modulator that competes with the known NPC1L1 modulator.

In specific embodiments, the saturation and measurement steps comprises: (a) contacting MDCK cells with increasing amounts of labeled known NPC1L1 modulator, (b) removing unbound, labeled known NPC1L1 modulator (e.g., by washing), and (c) measuring the amount of remaining bound, labeled NPC1L1 modulator.

In particular embodiments, the present invention relates to a method for identifying NPC1L1 modulators, which comprises (a) contacting MDCK cells bound to a known amount of labeled bound sterol (e.g., cholesterol) or 5α-stanol with a candidate NPC1L1 modulator; and (b) measuring the amount of labeled bound sterol or 5α-stanol; substantially reduced direct or indirect binding of the labeled sterol or 5α-stanol to NPC1L1 compared to what would be measured in the absence of the candidate NPC1L1 modulator indicating an NPC1L1 modulator.

The present invention additionally relates to methods for identifying and evaluating NPC1L1 modulators which comprises (a) incubating MDCK cells or a membrane fraction thereof with SPA beads (e.g., WGA coated YOx beads or WGA coated YSi beads) for a period of time sufficient to allow capture of the MDCK cells or membrane fraction by the SPA beads; (b) contacting the SPA beads obtained from step (a) with (i) detectably labeled known NPC1L1 modulator (e.g., labeled, known ligand or agonist or antagonist, including but not limited to ³H-cholesterol, ³H-ezetimibe, ¹²⁵I-ezetimibe or a ³⁵S-ezetimibe analog) and (ii) a candidate NPC1L1 modulator (or sample containing same); and (c) measuring fluorescence to determine scintillation; substantially reduced fluorescence as compared to that measured in the absence of the candidate NPC1L1 modulator indicating the candidate NPC1L1 modulator competes for binding with the known NPC1L1 modulator.

In alternative embodiments, the present invention relates to methods for identifying NPC1L1 modulators which comprises: (a) incubating MDCK cells or a membrane fraction thereof with SPA beads for a period of time sufficient to allow capture of the MDCK cells or membrane fraction by the SPA beads; (b) contacting the SPA beads obtained from step (a) with detectably labeled candidate NPC1L1 modulator; and (c) measuring fluorescence to detect the presence of a complex between the labeled candidate NPC1L1 modulator and the MDCK cell or membrane fraction expressing NPC1L1 or a complex including NPC1L1.

In related embodiments, the present invention relates to a method for identifying NPC1L1 modulators which comprises: (a) providing MDCK cells, lysate or membrane fraction of the foregoing bound to a plurality of support particles (e.g., in solution); said support particles impregnated with a fluorescer (e.g., yttrium silicate, yttrium oxide, diphenyloxazole and polyvinyltoluene); (b) contacting the MDCK cells, lysate or membrane fraction with a radiolabeled (e.g., with ³H, ¹⁴C or ¹²⁵I) known NPC1L1 modulator; (c) contacting the MDCK cells, lysate or membrane fraction with a candidate NPC1L1 modulator or sample containing same; and (d) comparing emitted radioactive energy with that emitted in a control not contacted with the candidate NPC1L1 modulator; wherein substantially reduced light energy emission, compared to that measured in the absence of the candidate NPC1L1 modulator indicates an NPC1L1 modulator.

In specific embodiments, the present invention relates to a method for identifying NPC1L1 modulators which comprises: (a) providing, in an aqueous suspension, a plurality of support particles attached to MDCK cells, lysate or membrane fraction of the foregoing, said support particles impregnated with a fluorescer; (b) adding, to the suspension, a radiolabeled (e.g., with ³H, ¹⁴C or ¹²⁵I) known NPC1L1 modulator; (c) adding, to the suspension, a candidate NPC1L1 modulator or sample containing same; and (d) comparing emitted radioactive energy emitted with that emitted in a control where the candidate NPC1L1 modulator was not added; wherein substantially reduced light energy emission, compared to what would be measured in the absence of the candidate NPC1L1 modulator indicates an NPC1L1 modulator.

In specific embodiments, the present invention relates to methods for identifying NPC1L1 modulators which comprises: (a) providing MDCK cells transfected to over-express NPC1L1; (b) reducing or depleting cholesterol from the plasma membrane of the cells (including, but not limited to, by providing methyl-β-cyclodextrin or by inhibiting or blocking endogenous cholesterol synthesis, for example, by providing a statin); (c) contacting MDCK cells with detectably labeled sterol (e.g., ³H-cholesterol or ¹²⁵I-cholesterol)) or 5α-stanol and a candidate NPC1L1 modulator; and (d) monitoring for an effect on cholesterol flux.

In additional embodiments, the present invention relates to methods of identifying NPC1L1 modulators which comprises: (a) providing MDCK cells transfected to over-express NPC1L1; (b) reducing or depleting cholesterol from the plasma membrane of the cells (including, but not limited to, by providing methyl-β-cyclodextrin or by inhibiting or blocking endogenous cholesterol synthesis, for example, by providing a statin); (c) contacting MDCK cells with detectably labeled sterol (e.g., ³H-cholesterol or ¹²⁵I-cholesterol)) or 5α-stanol; (d) providing to said MDCK cells a known NPC1L1 modulator, including but not limited to ezetimibe (“EZE”), analogs or functional equivalents thereof; (e) providing to said cells a candidate NPC1L1 modulator, and (f) and measuring NPC1L1-mediated sterol (e.g., cholesterol) or 5α-stanol uptake; a decrease in sterol or 5α-stanol uptake as compared to that effected in the absence of the candidate NPC1L1 modulator indicating an NPC1L1 antagonist; and an increase of sterol or 5α-stanol influx as compared to that effected in the absence of the candidate NPC1L1 modulator indicating an NPC1L1 agonist.

In specific embodiments, the present invention provides a method for identifying an NPC1L1 modulator capable of effecting NPC1L1-mediated cholesterol absorption or flux, which comprises: (a) providing MDCK cells transfected to over-express NPC1L1; (b) reducing or depleting cholesterol from the plasma membrane (e.g., by using methyl-β-cyclodextrin or through any suitable alternative means); (c) contacting the MDCK cells with detectably labeled sterol (e.g., cholesterol) or 5α-stanol; (d) providing a candidate NPC1L1 modulator to the MDCK cells; and (e) measuring uptake or influx of the detectably labeled sterol or 5α-stanol; a decrease in cholesterol influx upon the addition of the candidate NPC1L1 modulator indicating an NPC1L1 antagonist; and an increase in cholesterol influx indicating an NPC1L1 agonist. In specific embodiments, a cellular lysate is prepared between steps (d) and (e). In specific embodiments, detection of uptake of the detectably labeled sterol or 5α-stanol is measured by liquid scintillation counting of a cellular lysate. In additional embodiments, the method further comprises the administration of a known NPC1L1 modulator as a comparator or control.

In additional embodiments, the present invention provides a method for identifying an NPC1L1 modulator capable of effecting NPC1L1-mediated cholesterol absorption or flux, which comprises: (a) providing MDCK cells transfected or induced to express NPC1L1; (b) inhibiting or blocking endogenous cholesterol synthesis (e.g., with the HMG CoA reductase inhibitor lovastatin or by any suitable alternative means); (c) contacting the MDCK cells with detectably labeled sterol (e.g., cholesterol) or 5α-stanol; (d) providing a candidate NPC1L1 modulator to the MDCK cells; and (e) measuring uptake or influx of the detectably labeled sterol or 5α-stanol; a decrease in cholesterol influx upon the addition of the candidate NPC1L1 modulator indicating an NPC1L1 antagonist; and an increase in cholesterol influx indicating an NPC1L1 agonist. In specific embodiments, a cellular lysate is prepared between steps (d) and (e). In specific embodiments, detection of uptake of the detectably labeled sterol or 5α-stanol is measured by liquid scintillation counting of a cellular lysate. In additional embodiments, the method further comprises the administration of a known NPC1L1 modulator as a comparator or control.

The present invention further relates to isolated or purified canine NPC1L1 polypeptide wherein said polypeptide comprises SEQ ID NO: 5.

The present invention also relates to isolated nucleic acid encoding canine NPC1L1 polypeptide which comprises SEQ ID NO: 5. In particular embodiments, the isolated nucleic acid comprises SEQ ID NO: 4.

The present invention also encompasses vectors comprising the described nucleic acid encoding SEQ ID NO: 5 (or nucleic acid comprising SEQ ID NO: 4).

The present invention further encompasses, as particular embodiments hereof, cells, populations of cells, and non-human transgenic animals comprising the nucleic acid and vectors described herein. In particular aspect, the present invention encompasses MDCK cells expressing recombinant (i.e., derived by man) NPC1L1 protein including but not limited to that of SEQ ID NO: 5.

### Terms

Unless defined otherwise, technical and scientific terms used herein have the meanings commonly understood by one of ordinary skill in the art to which the present invention pertains. One skilled in the art will recognize other methods and materials similar or equivalent to those described herein, which can be used in the practice of the present teachings. It is to be understood, that the teachings presented herein are not intended to limit the methodology or processes described herein.

For purposes of the present invention, the following terms are defined below:

A “polynucleotide”, “nucleic acid” or “nucleic acid molecule” may refer to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in single stranded form, double-stranded form or otherwise.

A “polynucleotide sequence”, “nucleic acid sequence” or “nucleotide sequence” is a series of nucleotide bases (also called “nucleotides”) in a nucleic acid, such as DNA or RNA, and means any chain of two or more nucleotides.

A “coding sequence” or a sequence “encoding” an expression product, such as a RNA, polypeptide, protein, or enzyme, is a nucleotide sequence that, when expressed, results in production of the product.

The term “gene” means a DNA sequence that codes for or corresponds to a particular sequence of ribonucleotides or amino acids which comprise all or part of one or more RNA molecules, proteins or enzymes, and may or may not include regulatory DNA sequences, such as promoter sequences, which determine, for example, the conditions under which the gene is expressed. Genes may be transcribed from DNA to RNA which may or may not be translated into an amino acid sequence.

A “protein sequence”, “peptide sequence” or “polypeptide sequence” or “amino acid sequence” may refer to a series of two or more amino acids in a protein, peptide or polypeptide.

“Protein”, “peptide” or “polypeptide” includes a contiguous string of two or more amino acids.

“Isolated” as used herein describes a property as it pertains to the MDCK cells that makes it different from that found in nature. The difference may be, for example, that the cells are in a different environment than that found in nature or that the MDCK cells are those which are substantially free from other cell types.

The terms “isolated polynucleotide” or “isolated polypeptide” include a polynucleotide (e.g., RNA or DNA molecule, or a mixed polymer) or a polypeptide, respectively, which are partially or fully separated from other components that are normally found in cells or in recombinant DNA expression systems. These components include, but are not limited to, cell membranes, cell walls, ribosomes, polymerases, serum components and extraneous genomic sequences.

An isolated polynucleotide or polypeptide will, preferably, be an essentially homogeneous composition of molecules but may contain some heterogeneity.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an “expression product” such as an RNA (e.g., mRNA) or a protein. The expression product itself may also be said to be “expressed” by the cell.

The term “functional equivalent thereof” means that the protein, compound, biological or other exhibits at least 10% and in order of increasing preference, 20%, 30%, 40%, 50%, 60%, 70,%, 80%, 90%, or 95% of the activity of that referred to. For purposes of exemplification, with respect to, for example, EZE or its derivatives, the activity could be either specific binding to NPC1L1 or inhibition of NPC1L1-mediated absorption of cholesterol, or both. In another example, in terms of a functional equivalent of NPC1L1, the activity could be specific binding to EZE, its derivatives (or other previously characterized NPC1L1 modulators), or the absorption of cholesterol. In specific examples, the activity may be the absorption of cholesterol in an EZE-sensitive manner (i.e., where the absorption of cholesterol is significantly reduced in the presence of EZE).

The twit “selective” or “specific” with respect to binding refers to the fact that the protein, compound, biological or other does not show significant binding to other than the particular substance or protein, except in those specific instances where the protein, compound, biological or other is manipulated to, or possesses, an additional, distinct specificity to other than the particular substance or protein. This may be the case, for instance, with bispecific or bifunctional molecules where the molecule is designed to bind or effect two functions, at least one of which is to specifically affect the particular substance or protein. Furthermore, “specific binding” includes direct or indirect binding directly to the particular substance or protein. Indirect binding may happen, for example, when the particular substance or protein is presented via another moiety such as a complex. The determination of specific binding may be made by comparing with a negative control.

“Candidate cholesterol modulator”, “candidate NPC1L1 modulator”, “sample”, “candidate compound” or “candidate substance” refers to a compound, biologic, protein, composition or other which is evaluated in a test or assay, for example, for the ability to bind to NPC1L1, induce NPC1L1-mediated cholesterol uptake into the cell and/or induce cholesterol homeostasis within the cell. The composition may comprise candidate compounds, such as small molecules, peptides, nucleotides, polynucleotides, subatomic particles (e.g., a particles, f3 particles) or antibodies.

As used herein, the term “sterol” includes, but is not limited to, cholesterol and phytosterols (including, but not limited to, sitosterol, campesterol, stigmasterol and avenosterol). As used herein, the term “5α-stanol” includes, but is not limited to, cholestanol, 5α-campestanol and 5α-sitostanol.

### BRIEF DESCRIPTION OF THE DRAWINGS

FIG. 1A illustrates saturation studies of [³H]AS binding to HEK 293 cells stably transfected with rat NPC1L1 (“rat NPC1L1/HEK293 cells”). rNPC1L1/HEK293 cells were seeded in 96-well poly-D-lysine plates, at a density of 10,000 cells/well and incubated with increasing concentrations of [³H]AS for 4 hours at 37° C. Bound radioligand was separated from free radioligand. Total binding (▴), non-specific binding determined in the presence of 100 μM ezetimibe glucuronide (“EZE-gluc”) () and specific binding (▪), defined as the difference between total and nonspecific binding are presented. Specific binding was a saturable function of [³H]AS (see Example 1) concentration and displayed a single high affinity site with K_{d} of 4.62 nM and Bₘₐₓ of 2.21×10⁶ sites/cell.

FIG. 1B illustrates association kinetics of [³H]AS binding to rNPC1L1/HEK293 cells. rNPC1L1/HEK293 cells were incubated with 5 nM [³H]AS at 37° C. Nonspecific binding determined in the presence of 100 μM EZE-gluc was time invariant and has been subtracted from experimental points. Inset: a semilogarithmic representation of the pseudo-first order association reaction, where Bₑ and Bₜ represent ligand bound at equilibrium (e) and time (t), respectively, yielded k_{obs} (0.0208 min⁻¹), corresponding to a k₁ of kₒₙ (0.0024 nM⁻¹ min⁻¹).

FIG. 1C illustrates dissociation kinetics of [³H]AS binding to rNPC1L1/HEK293 cells. After incubation with 5 nM [³H]AS overnight, wells were rinsed and rNPC1L1/HEK293 cells were incubated with growth media containing 100 μM EZE-gluc for different amounts of time at 37° C. [³H]AS dissociation followed mono-exponential kinetics, indicative of a first-order reaction with k_{off}=0.0059 min⁻¹. The K_{d} determined from k_{off}/kₒₙ is 2.46 nM.

FIG. 2A illustrates pharmacology data concerning the interaction of cell surface rat NPC1L1 with [³H]AS. rNPC1L1 cells were incubated with 5.36 nM [³H]AS in the presence or absence of increasing concentrations of AS, PS (see Example 1), EZE-gluc or ezetimibe (“EZE”) for 4 hours at 37° C. Inhibition of binding was assessed relative to an untreated control. Specific binding was fit to a single-site inhibition model, yielding IC₅₀ values of (▪) 5.25 nM (AS), (♦) 6.61 nM (PS), (▴) 398 nM (EZE) and () 182 nM (EZE-gluc).

FIG. 2B illustrates acid wash data concerning the interaction of cell surface rat NPC1L1 with [³H]AS. Plot shows the normalized equilibrium levels of bound radioligand to rNPC1L1/HEK293 cells after 2 hours incubation with 5 nM [³H]AS (1B, 5B, 15B). After washing the cells once with PBS, the cells were acid washed by incubation in DMEM pH 3.5 for 1 (1A), 5 (5A), or 15 (15A) minutes. Thereafter, acid was removed by two PBS washes and after re-presentation of 5 nM [³H]AS for 2 hours, radioligand binding is monitored for each acid wash condition.

FIG. 3A illustrates an equilibrium determination of 5 nM [³H]AS binding to selected cell lines. At the appropriate time after seeding, binding was measured at 37° C. for 4 hours in the absence or presence of 100 μM EZE-gluc.

FIG. 3B illustrates saturation binding data for [³H]AS binding to MDCKII cells. MDCKII cells were seeded into tissue culture treated 96-well plates, at a density of 25,000 cells/well and incubated with increasing concentrations of [³H]AS for 4 hours at 37° C. Bound radioligand was separated from free radioligand. Total binding (), non-specific binding determined in the presence of 100 μM EZE-gluc (▪) and specific binding (▴), defined as the difference between total and nonspecific binding are presented. Specific binding was a saturable function of [³H]AS concentration and displayed a single high affinity site with K_{d} of 0.59 nM and Bₘₐₓ of 4.9×10⁵ sites/cell.

FIG. 4A illustrates association kinetics for [³H]AS binding to MDCKII cells. Cells were incubated with 1.2 nM [³H]AS for indicated amounts of time at 37° C. Nonspecific binding determined in the presence of 100 μM EZE-gluc was time invariant and has been subtracted from experimental points. Inset: a semilogarithmic representation of the pseudo-first order association reaction, where Bₑ and Bₜ represent ligand bound at equilibrium (e) and time (t), respectively, yielded k_{obs} (0.0247 min⁻¹), corresponding to a k₁ of kₒₙ (0.0163 nM⁻¹ min⁻¹).

FIG. 4B illustrates dissociation kinetics for [³H]AS binding to MDCKII cells. After incubation with 1 nM [³H]AS overnight, wells were rinsed and cells were incubated with growth media containing 100 μM EZE-gluc for different amounts of time at 37° C. [³H]AS dissociation followed mono-exponential kinetics, indicative of a first-order reaction with k_{off}=0.0023 min⁻¹. The K_{D} determined from k_{off}/kₒₙ is 0.14 nM.

FIG. 4C illustrates acid wash data for [³]AS binding to MDCKII cells. Plot shows the normalized equilibrium levels of bound radioligand to MDCKII cells after 2 hours incubation with 5 nM [³H]AS (1B, 5B, 15B). After washing the cells once with PBS, the cells were acid washed by incubation in DMEM pH 3.5 for 1 (1A), 5 (5A) or 15 minutes (15A). Thereafter, acid was removed by two PBS washes and after re-presentation of 5 nM [³H]AS for 2 hours, radioligand binding was monitored for each acid wash condition (1PA, 5PA, 15PA).

FIG. 4D illustrates NPC1L1-like activity expressed at the apical membrane of MDCKII cells. MDCKII cells were presented with 1 nM [³H]AS at either the apical (a) or basolateral (b) side of cells grown on impermeable Transwells in the absence (T) or presence (NS) of 100 μM EZE-gluc.

FIG. 4E illustrates the pharmacology of [³H]AS binding to MDCKII cells. Cells were incubated with 5.49 nM [³H]AS in the presence or absence of increasing concentrations of AS, PS, EZE-gluc or EZE for 4 hours at 37° C. Inhibition of binding was assessed relative to an untreated control. Specific binding was fit to a single-site inhibition model, yielding IC₅₀ values of (▪) 2.86 nM (AS), (♦) 3.02 nM (PS), (▴) 126 nM (EZE) and () 24 nM (EZE-gluc).

FIG. 5 illustrates the pharmacology of [³H]AS binding to dog NPC1L1 transiently expressed in TsA201 cells. Cells were incubated with 4.65 nM [³H]AS in the presence or absence of increasing concentrations of AS, PS, EZE-gluc or EZE for 4 hours at 37° C. Inhibition of binding was assessed relative to an untreated control. Specific binding was fit to a single-site inhibition model, yielding IC₅₀ values of (▪) 3.79 nM (AS), (♦) 3.73 nM (PS), (▴) 111 nM (EZE) and () 27 nM (EZE-gluc). Inset: PCR product of full length dog NPC1L1 cDNA.

FIG. 6A illustrates a time course of 5 nM [³H]AS binding to MDCKII cells grown in either 10% FBS or 5% LPDS in the absence or presence of 4 μM lovastatin. At each time point, cells are harvested and [³H]AS binding determined in the absence (T) or presence (NSB) of 100 μM EZE-gluc. Subtraction of the non-specific binding from the total binding yields the plotted specific [³H]AS binding.

FIG. 6B illustrates how Lovastatin leads to an increase in [³H]AS binding to MDCKII cells grown in 5% LPDS. FIG. 6B particularly illustrates saturation binding of [³H]AS to MDCKII cells three days after initiating growth in either 5% LPDS or 5% LPDS with 4 μM lovastatin. Specific binding is shown and was assessed from the difference of total and non-specific binding (defined with 100 μM EZE-gluc). Binding was measured with 25000 cells in a volume of 200 μl after 2 hours incubation at 37° C. Data were fit by nonlinear regression. Binding data identify a single high affinity site with K_{D}=180 pM and Bₘₐₓ of either 75 pM (5% LPDS) or 154 pM (5% LPDS and 4 μM lovastatin).

FIG. 7A illustrates results from a functional assay of [³H] sterol influx into MDCKII-Flp cells overexpressing human NPC1L1. FIG. 7A particularly illustrates a correlation of human NPC1L1 expression levels with PS blockade [³H] cholesterol “[³H]Ch” influx into MDCKII-Flp cells and human NPC1L1 variants. I, Influence of pmCD and PS on the influx of [³H]Ch into MDCKII-Flp cells. Cells were seeded on 96-well plates and [³H]Ch flux was performed. Cells were pre-incubated in the absence or presence of 10 μM PS for 3 hours. Thereafter, cells were incubated with or without 5.5% βmCD for 45 minutes prior to addition of [³H]cholesterol in 5% LPDS. II, Binding of [³H]AS to MDCKII-Flp and human NPC1L1/MDCK II-Flp cells. MDCKII-Flp and hNPC1L1/MDCKII-Flp cells were seeded on 96-well plates. Cells were incubated with increasing concentrations of [³H]AS for 4 hours at 37° C. Bound radioligand was separated from free radioligand. Specific binding was fit to a single-site saturation model, yielding K_{d}/Bₘₐₓ values of 0.4 nM/73 pM for MDCKII-Flp cells (▪) and 11 nM/1260 pM for hNPC1L1/MDCKII-Flp cells (). III, Influence of βmCD and PS on the influx of [³H]Ch into human NPC1L1/MDCKII-Flp cells. Cells were seeded on 96-well plates and [³H]Ch flux was performed. Cells were pre-incubated in the absence or presence of 10 μM PS for 3 hours. Thereafter, cells were incubated with or without 5.5% βmCD for 45 minutes prior to addition of [³H] cholesterol in 5% LPDS.

FIG. 7B illustrates results from a functional assay of [³H] sterol influx into MDCKII-Flp cells overexpressing dog NPC1L1. FIG. 7B particularly illustrates a correlation of dog NPC1L1 expression levels with PS blockade [³H]cholesterol influx into MDCKII-Flp cells and dog variants. I, Influence of βmCD and PS on the influx of [³H]Ch into dNPC1L1/MDCKII-Flp cells. Cells were seeded on 96-well plates and [³H]Ch flux was performed. Cells were pre-incubated in the absence or presence of 10 μM PS for 3 hours. Thereafter, cells were incubated with or without 5.5% LPDS. II, Binding of [³H]AS to dog NPC1L1/MDCKII-Flp cells before and after induction. Dog NPC1L1/MDCKII-Flp cells were seeded on 96-well plates. Cells were incubated with increasing concentrations of [³H]AS for 4 hours at 37° C. Bound radioligand was separated from free radioligand. Specific binding was fit to a single-site saturation model, yielding K_{d}/Bₘₐₓ values of 0.78 nM/131 pM for dNPC1L1/MDCKII-Flp cells without induction (▪) and 1.53 nM/384 pM for cells after 24 hours induction with 4 mM sodium butyrate (▴). III, Influence of βmCD and PS on the influx of [³H]Ch into dog NPC1L1/MDCKII-Flp cells. Cells were seeded on 96-well plates, dog NPC1L1 was induced for 24 hours with 4 mM sodium butyrate and [³H]Ch flux was performed. Cells were pre-incubated in the absence or presence of 10 μM PS for 3 hours. Thereafter, cells were incubated with or without 5.5% βmCD for 45 minutes prior to addition of [³H] cholesterol in 5% LPDS.

FIG. 7C illustrates results from a functional assay of [³H] sterol influx into MDCKII-Flp cells overexpressing dog or human NPC1L1. FIG. 7C particularly illustrates compound blockade [³H] Cholesterol flux into dog NPC1L1/MDCKII-Flp and human NPC1L1/MDCKII-Flp cells. Dog NPC1L1/MDCKII-Flp and human MDCKII-Flp cells were seeded and treated. Cholesterol flux was performed in the presence of increasing concentrations of PS. [³H]Ch flux was fit to a single-site inhibition model, yielding IC₅₀ values of (▪) 0.32 nM for dNPC1L1/MDCKII-Flp and () 10.3 nM for hNPC1L1/MDCKII-Flp.

FIG. 7D illustrates results of characterized compounds' ability to bind to and block [³H] sterol flux through MDCKII-Flp cells overexpressing human NPC1L1. FIG. 7D particularly illustrates a correlation between a compound's affinity for human NPC1L1 and its ability to block cholesterol flux. Binding and flux experiments were performed. Specific [³H]AS was fit to a single-site inhibition model, yielding Kᵢ values of () 5 nM (PS), (▴) 209 nM (EZE-gluc), (♦) 1.3 μM (EZE), and (▪) N.D. (ent-1). [³H]Ch flux was fit to a single-site inhibition model yielding IC₅₀ values of () 7 nM (PS), (▴) 300 nM (EZE-gluc), (♦)>1 μM (EZE), and (▪) N.D. (ent-1).

### DETAILED DESCRIPTION OF THE INVENTION

The present invention relates to a novel method for using polarized Madin-Darby Canine Kidney (“MDCK”) cells in the study and identification of cholesterol modulators.

Applicants have surprisingly found that MDCK cells exhibit cholesterol-sensitive endogenous expression of a critical cholesterol absorption protein, NPC1L1 in the apical membrane of MDCK cells, in a similar manner to enterocytes despite the fact that they originate from a different organ. Based on the foregoing, they are expected to possess all of the necessary proteins for cholesterol flux across the apical membrane. This biochemically tractable source of critical cholesterol-regulating factors is of great utility in providing a mechanistic insight into cholesterol absorption pathways and presents a viable system to identify and evaluate novel cholesterol modulators.

Accordingly, the present invention relates to the use of MDCK cells for use in the evaluation of cholesterol modulators (i.e., compounds, biologicals and other molecules that impact cholesterol homeostasis through an effect on cholesterol absorption, transport, synthesis and/or catabolism). In additional embodiments, the present invention relates to the use of MDCK cells for use in the identification and study of cellular proteins or factors involved in the regulation of cholesterol absorption.

### Application in the Study of NPC1L1

NPC1L1 is a protein which mediates the absorption of dietary cholesterol in the proximal region of the intestine. NPC1L1 is a validated target for lowering low density lipoprotein cholesterol, and inhibitors thereof are effectively used in the treatment of hypercholesterolemia. NPC1L1 is particularly sensitive to the cholesterol absorption inhibitor ezetimibe (“EZE”), alone or in combination with a statin.

The molecular mechanism of NPC1L1-dependent cholesterol absorption in the intestine remains unclear. Therefore, the identification and validation of a cell line expressing endogenous NPC1L1 in a cholesterol-sensitive manner would permit detailed studies into the process of NPC1L1-dependent cholesterol flux.

Polarized, epithelial MDCK cells were identified as expressing robust amounts of an NPC1L1-like activity with similar pharmacology to rat NPC1L1. Furthermore, and in agreement with a recent study comparing the binding of glucuronidated ezetimibe to multiple species of NPC1L1 orthologs (Hawes et al., 2007 Mol. Pharmacol. 71:19-29), MDCKII cells were found to consistently bind EZE analogs more potently than rat NPC1L1 expressed in HEK293 cells. Importantly, [³H]AS binding to MDCKII cells occurs almost exclusively at the apical surface, consistent with the apparent localization of NPC1L1 in both enterocytes (Altmann et al., 2004 Science 303:1201-1204 and hepatocytes (Yu et al., 2006 J. Biol. Chem. 281:6616-6624). This presented a workable in vitro system for detailed biochemical studies of NPC1L1 function.

Accordingly, the present invention relates to the use of MDCK cells to evaluate the functioning of NPC1L1 and modulators thereof (i.e., compounds, biologicals and other molecules that specifically impact the functioning of NPC1L1 in cholesterol absorption, including but not limited to the antagonism or agonism of NPC1L1-mediated cholesterol influx). NPC1L1 modulators may be useful in the treatment and management of a variety of medical conditions, including elevated serum sterol (e.g., cholesterol) or 5α-stanol.

### NPC1L1 Binding Assays

The present invention relates to the use of MDCK cells in an assay to detect NPC1L1 modulators that can bind to NPC1L1 and impact the functioning of NPC1L1 in cholesterol influx. In specific embodiments, the method comprises contacting MDCK cells with a candidate NPC1L1 modulator and identifying those candidate NPC1L1 modulators that specifically bind to NPC1L1. Such experiments may be performed along with a control experiment wherein NPC1L1-dependent binding is minimal or absent, including but not limited to a different cell line not expressing NPC1L1, cells from which genomic NPC1L1 DNA has been disrupted or deleted, or cells where endogenous NPC1L1 RNA has been depleted, for example, by RNAi.

In specific embodiments, the present invention relates to a method which comprises contacting the MDCK cells with a detectably labeled known or previously characterized NPC1L1 modulator, and a candidate NPC1L1 modulator, and determining whether the candidate modulator binds to NPC1L1, displacing the detectably labeled NPC1L1 modulator, essentially competing for binding with the known NPC1L1 modulator. This is typically measured after removing unbound, labeled ligand or known antagonist or agonist by washing. Where the candidate NPC1L1 modulator competes with the known NPC1L1 modulator, the candidate NPC1L1 modulator binds NPC1L1 selectively and is a likely inhibitor of sterol (e.g., cholesterol) and 5α-stanol absorption. One measure of competition with a known NPC1L1 modulator is reduced binding of the known NPC1L1 modulator to NPC1L1, compared to what would be measured in the absence of the candidate modulator.

“Known” or “previously characterized” NPC1L1 modulators, as such terms are used interchangeably herein, are compounds, biologicals, proteins or other which have been determined to be either ligand, agonists or antagonists of NPC1L1-mediated activity. Said known NPC1L1 modulators include but are by no means limited to sterols (such as cholesterol, phytosterols, including, but not limited to, sitosterol, campesterol, stigmasterol and avenosterol), cholesterol oxidation products, 5α-stanol (including, but not limited to, cholestanol, 5α-campestanol and 5α-sitostanol), substituted azetidinone (e.g., ezetimibe (“EZE”)), BODIPY-ezetimibe (Altmann et al., 2002 Biochim. Biophys. Acta 1580(1): 77-93) or 4″, 6″-bis[(2-fluorophenyl)carbamoyl]-beta-D-cellobiosyl derivative of 11-ketotigogenin as described in DeNinno, et al., (1997) (J. Med. Chem. 40(16): 2547-54) or any substituted azetidinone, analogs or functional equivalents thereof. Non-limiting examples of suitable substituted azetidinones for use in the assays disclosed herein include but are not limited to those disclosed in U.S. Pat. Nos. RE37,721; 5,631,365; 5,767,115; 5,846,966; 5,688,990; 5,656,624; 5,624,920; 5,698,548; 5,756,470; 5,688,787; 5,306,817; 5,633,246; 5,627,176; 5,688,785; 5,744,467; 5,846,966; 5,728,827; 6,632,933, U.S. Patent Publication No 2003/0105028 and U.S. Patent Publication No. 2007/0078098. Specific embodiments are wherein the known NPC1L1 modulator is substituted 2-azetidinone, and preferably substituted 2-azetidinone-glucuronide. Substituted 2-azetidinones including but not limited to substituted 2-azetidinone-glucuronide, are disclosed in International Publication No. WO 2005/069900, U.S. Pat. No. 5,756,470, International Publication No. WO 02/066464 and US Publication No. US 2002/0137689. Ezetimibe can be prepared by a variety of methods well know to those skilled in the art, for example such as are disclosed in U.S. Pat. Nos. 5,631,365, 5,767,115, 5,846,966, 6,207,822, U.S. Patent Application Publication No. 2002/0193607 and PCT Patent Application WO 93/02048. In preferred embodiments, Ezetimibe or its derivatives are glucoronidated. Particular embodiments are wherein the known NPC1L1 modulator has a binding affinity K_{D} value of 200 nM or lower and, in further specific embodiments, 100 nM, 50 nM, and 10 nM or lower.

Known modulators, as one of skill in the art is aware, may be labeled with any label which enables the modulator to be specifically detected through either its' presence, binding and/or activity, as appropriate. Examples of labels of use in the disclosed methods include, but are not limited to, ³H, ³⁵S, ¹²⁵I, ³²P, ¹⁴C, biotin, or fluorescent labels. Various labeled forms of sterols (e.g., cholesterol) or 5α-stanols are available commercially or can be generated using standard techniques (e.g., Cholesterol-[1,2-³H(N)], Cholesterol-[1,2,6,7-³H(N)] or Cholesterol-[7-³H(N)]; American Radiolabeled Chemicals, Inc.; St. Louis, Mo.), In a preferred embodiment, ezetimibe is fluorescently labeled with a BODIPY group (Altmann, et al., 2002, Biochim Biophys. Acta 1580(I):77-93) or labeled with a detectable group such as ³⁵S, ¹²⁵I, or ³H, and preferably, ³⁵S.

### Saturation Analysis

The present invention also relates to methods for identifying NPC1L1 modulators which comprises: (a) saturating NPC1L1 binding sites on MDCK cells with a detectably labeled previously characterized NPC1L1 modulator, (b) measuring the amount of bound label, (c) contacting the cells with an unlabeled candidate NPC1L1 modulator (or, in the alternative, a candidate modulator bearing a distinct label); and (d) measuring the amount of bound label remaining; displacement of the label indicating the presence of an NPC1L1 modulator that competes with the known NPC1L1 modulator.

In specific embodiments, the saturation and measurement steps comprises: (a) contacting MDCK cells with increasing amounts of labeled known NPC1L1 modulator, (b) removing unbound, labeled known NPC1L1 modulator (e.g., by washing), and (c) measuring the amount of remaining bound, labeled NPC1L1 modulator. As the amount of the labeled NPC1L1 modulator is increased, a point is eventually reached at which all binding sites are occupied or saturated. Specific binding of the labeled NPC1L1 modulator is abolished by a large excess of unlabeled NPC1L1 modulator.

Preferably, an assay system is used in which non-specific binding of the labeled NPC1L1 to the receptor is minimal. Non-specific binding is typically less than 50%, preferably less than 15%, more preferably less than 10% and, most preferably, 5% or less of the total binding of the labeled ligand or known antagonist or agonist.

In particular embodiments, the present invention relates to a method for identifying NPC1L1 modulators, which comprises (a) contacting MDCK cells bound to a known amount of labeled bound sterol (e.g., cholesterol) or 5α-stanol with a candidate NPC1L1 modulator; and (b) measuring the amount of labeled bound sterol or 5α-stanol; substantially reduced direct or indirect binding of the labeled sterol or 5α-stanol to NPC1L1 compared to what would be measured in the absence of the candidate NPC1L1 modulator indicating an NPC1L1 modulator.

This assay can include a control experiment lacking any NPC1L1-dependent ligand (e.g., sterol such as cholesterol or 5α-stanol) binding, for example, including but not limited to a different cell line not expressing NPC1L1, cells from which genomic NPC1L1 DNA has been disrupted or deleted, or cells where endogenous NPC1L1 RNA has been depleted, for example, by RNAi.

In specific embodiments, the labeled ligand employed in any of the assays disclosed herein may be obtained by labeling a sterol (e.g., cholesterol) or a 5α-stanol or a known NPC1L1 agonist or antagonist with a measurable group (e.g., ³⁵S, ¹²⁵I or ³H). In addition, various labeled forms of sterols (e.g., cholesterol) or 5α-stanols are available commercially or can be generated using standard techniques (e.g., Cholesterol-[1,2-³H(N)], Cholesterol-[1,2,6,7-3H(N)] or Cholesterol-[7-³H(N)]; American Radiolabeled Chemicals, Inc; St. Louis, Mo.). In a preferred embodiment, ezetimibe is fluorescently labeled with a BODIPY group (Altmann, et al., (2002) Biochim. Biophys. Acta 1580(1): 77-93) or labeled with a detectable group such as ³⁵S, ¹²⁵I or ³H.

### SPA Binding Assays

NPC1L1 modulators may also be identified using scintillation proximity assays (SPA). SPA assays are conventional and very well known in the art; see, for example, U.S. Pat. No. 4,568,649. In SPA-type assays, the target of interest is immobilized to a small microsphere approximately 5 microns in diameter. The microsphere, typically, includes a solid scintillant core which has been coated with a polyhydroxy film, which in turn contains coupling molecules, which allow generic links for assay design. When a radioisotopically labeled molecule binds to the microsphere, the radioisotope is brought into close proximity to the scintillant and effective energy transfer from electrons emitted by the isotope will take place resulting in the emission of light. While the radioisotope remains in free solution, it is too distant from the scintillant and the electron will dissipate the energy into the aqueous medium and therefore remain undetected. Scintillation may be detected with a scintillation counter. In general, 3H, ¹²⁵I and ³⁵S labels are well suited to SPA, although as the skilled artisan will no doubt be aware, any suitable label may be utilized.

The present invention, therefore, relates in specific embodiments to methods for identifying and evaluating NPC1L1 modulators which comprises (a) incubating MDCK cells or a membrane fraction thereof with SPA beads (e.g., WGA coated YOx beads or WGA coated YSi beads) for a period of time sufficient to allow capture of the MDCK cells or membrane fraction by the SPA beads; (b) contacting the SPA beads obtained from step (a) with (i) detectably labeled known NPC1L1 modulator (e.g., labeled, known ligand or agonist or antagonist, including but not limited to ³H-cholesterol, ³H-ezetimibe, ¹²⁵I-ezetimibe or a ³⁵S-ezetimibe analog) and (ii) a candidate NPC1L1 modulator (or sample containing same); and (c) measuring fluorescence to determine scintillation; substantially reduced fluorescence as compared to that measured in the absence of the candidate modulator indicating the candidate NPC1L1 modulator competes for binding with the known NPC1L1 modulator.

A control employing a blank (e.g., water) in place of the candidate NPC1L1 modulator may be used for purposes of comparing. In such a case, the amount of fluorescence measured would be compared with that measured in the absence of the candidate NPC1L1 modulator (i.e., that obtained with the blank).

In alternative embodiments, the present invention relates to methods for identifying NPC1L1 modulators which comprises: (a) incubating MDCK cells or a membrane fraction thereof with SPA beads for a period of time sufficient to allow capture of the MDCK cells or membrane fraction by the SPA beads; (b) contacting the SPA beads obtained from step (a) with detectably labeled candidate NPC1L1 modulator; and (c) measuring fluorescence to detect the presence of a complex between the labeled candidate NPC1L1 modulator and the MDCK cell or membrane fraction expressing NPC1L1 or a complex including NPC1L1. A candidate NPC1L1 modulator which binds directly or indirectly to NPC1L1 may possess NPC1L1 agonistic or antagonistic activity. As above, the assay may be performed along with a control experiment lacking or minimally possessing any NPC1L1-dependent binding. Said control experiment may be performed, for example, with a cell or cell membrane lacking any functional NPC1L1 including but not limited to a different cell line not expressing NPC1L1, cells from which genomic NPC1L1 DNA has been disrupted or deleted, or cells where endogenous NPC1L1 RNA has been depleted, for example, by RNAi. When a control experiment is performed, the level of binding observed in the presence of sample being tested for the presence of an antagonist may be compared with that observed in the control experiment.

In specific embodiments employing a SPA assay for identification and evaluation of NPC1L1 modulators, lectin wheat germ agglutinin (WGA) may be used as the SPA bead coupling molecule (Amersham Biosciences; Piscataway, N.J.). The WGA coupled bead captures glycosylated, cellular membranes and glycoproteins and has been used for a wide variety of receptor sources and cultured cell membranes. The binding protein is immobilized onto the WGA-SPA bead and a signal is generated on binding of an isotopically labeled ligand. Other coupling molecules which may be useful for SPA binding assays include poly-L-lysine and WGA/polyethyleneimine (Amersham Biosciences; Piscataway, N.J.). See, for example, Berry, J. A., et al., (1991) Cardiovascular Pharmacol. 17 (Supp1.7): S143-S145; Hoffman, R., et al., (1992) Anal. Biochem. 203: 70-75; Kienhus, et al., (1992). J. Receptor Research 12: 389-399; Jing, S., et al., (1992) Neuron 9: 1067-1079.

The scintillant contained in SPA beads may include, for example, yttrium silicate (YSi), yttrium oxide (YOx), diphenyloxazole or polyvinyltoluene (PVT) which acts as a solid solvent for diphenylanthracine (DPA).

### General Support Binding Assays

In related embodiments, the present invention relates to a method for identifying NPC1L1 modulators which comprises: (a) providing MDCK cells, lysate or membrane fraction of the foregoing bound to a plurality of support particles (e.g., in solution); said support particles impregnated with a fluorescer (e.g., yttrium silicate, yttrium oxide, diphenyloxazole and polyvinyltoluene); (b) contacting the particles with a radiolabeled (e.g., with ³H, ¹⁴C or ¹²⁵I) known NPC1L1 modulator; (c) contacting the particles with a candidate NPC1L1 modulator or sample containing same; and (d) comparing emitted radioactive energy with that emitted in a control not contacted with the candidate NPC1L1 modulator; wherein substantially reduced light energy emission, compared to what would be measured in the absence of the candidate NPC1L1 modulator indicates an NPC1L1 modulator. This is because the radiolabel emits radiation energy capable of activating the fluorescer upon the binding of the radiolabeled known NPC1L1 modulator to the polypeptide to produce light energy. Radiolabeled known NPC1L1 modulator that does not bind to the polypeptide is, generally, too far removed from the support particles to enable the radioactive energy to activate the fluorescer.

In specific embodiments thereof, the present invention relates to a method for identifying NPC1L1 modulators which comprises: (a) providing, in an aqueous suspension, a plurality of support particles attached to MDCK cells (lysate or membrane fractions thereof), said support particles impregnated with a fluorescer; (b) adding, to the suspension, a radiolabeled (e.g., with ³H, ¹⁴C or ¹²⁵I) known NPC1L1 modulator; (c) adding, to the suspension, a candidate NPC1L1 modulator or sample containing same; and (d) comparing emitted radioactive energy emitted with that emitted in a control where the candidate NPC1L1 modulator was not added; wherein substantially reduced light energy emission, compared to what would be measured in the absence of the candidate NPC1L1 modulator indicates an NPC1L1 modulator.

### Functional Assays

MDCK cells have been validated as an appropriate surrogate system for monitoring NPC1L1 function and, as exemplified herein, clearly possess required critical cellular factors necessary for cholesterol absorption. More specifically, Applicants evaluated and identified the ability of MDCK cells to perform EZE-sensitive cholesterol flux using a protocol described in the art; see, Yu et al., 2006 J. Biol. Chem., 281:6616-6624. Importantly, over-expression of NPC1L1 in MDCK cells resulted in cholesterol influx and the influx was pharmacologically modulated by known NPC1L1 modulators, such as ezetimibe (“EZE”) and its analogs. Over-expression of NPC1L1 into these cells afforded a considerable window for cholesterol flux that was capable of being pharmacologically modulated by EZE and its analogs, a window that was not readily apparent from MDCK cells in the absence of such manipulation. Over-expression of either human or dog NPC1L1 significantly effected the measurements of EZE-sensitive [³H] cholesterol flux as a consequence of the dramatic increase in levels of NPC1L1. In particular, Applicants found that, dependent on the species of NPC1L1, overexpression to a level such that there are at least 1,500,000 binding sites per cell provides a significant window to identify and measure cholesterol flux. This calculation, as well as the appropriate degree of expression for the assay of interest, may be readily determined by one of ordinary skill in the art using suitable methodology. One specific means to carry out this analysis upon measuring radiolabeled sterol flux is via the following protocol: starting with the Y-axis value reached at plateau, (1) convert counts per minute of radioactivity (“CPM”) to disintegrations per minute of radioactivity (“DPM”) to correct for liquid scintillation counting efficiency; (2) convert DPM to Ci; (3) correct for specific activity of radioligand in Ci/mmol; (4) convert into nM binding sites (5) divide by the number of cells/well.

The present invention, therefore, relates to the use of MDCK cells to identify NPC1L1 modulators that antagonize cholesterol influx or, alternatively, serve to further promote or aggravate cholesterol influx. In specific embodiments, said methods may employ known NPC1L1 modulators, including but not limited to ezetimibe (“EZE”), analogs or functional equivalents thereof as comparators or to establish the baseline (i.e., serve as a control). In specific embodiments, the known NPC1L1 modulator is azetidinone (e.g., ezetimibe) or an EZE-like compound including but not limited to [³]AS.

In specific embodiments, the present invention relates to methods for identifying NPC1L1 modulators which comprises: (a) contacting MDCK cells with detectably labeled sterol (e.g., ³H-cholesterol or ¹²⁵I-cholesterol)) or 5α-stanol and a candidate NPC1L1 modulator; and (b) monitoring for an effect on cholesterol flux. After an optional incubation, the cells may be washed to remove unabsorbed sterol or 5α-stanol. Remaining bound sterol or 5α-stanol may then be measured by detecting the presence of labeled sterol or 5α-stanol in the MDCK cells. In specific embodiments, assayed cells, lysates or fractions thereof (e.g., fractions resolved by thin-layer chromatography) may be contacted with a liquid scintillant and scintillation can be measured using a scintillation counter. Preferred methods in accordance herewith further comprise reducing or depleting cholesterol from the plasma membrane of the cells prior to step (a).

In the functional assays provided, preferably the sterol or 5α-stanol is attached to or delivered with a compound, molecule or agent that facilitates delivery of the sterol or stanol into and through the membrane lipid. In specific embodiments, the sterol or 5α-stanol is delivered with BSA; see, e.g., Yu et al., 2006 J. Biol. Chem. 281:6616-6624.

In additional embodiments, the present invention relates to methods of identifying NPC1L1 modulators which comprises: (a) contacting MDCK cells with detectably labeled sterol (e.g., ³H-cholesterol or ¹²⁵I-cholesterol)) or 5α-stanol; (b) providing to said MDCK cells a known NPC1L1 modulator, including but not limited to ezetimibe (“EZE”), analogs or functional equivalents thereof; (c) providing to said cells a candidate NPC1L1 modulator, and (d) and measuring NPC1L1-mediated sterol (e.g., cholesterol) or 5α-stanol uptake; a decrease in sterol or 5α-stanol uptake as compared to that effected in the absence of the candidate NPC1L1 modulator indicating an NPC1L1 antagonist; and an increase of sterol or 5α-stanol influx as compared to that effected in the absence of the candidate NPC1L1 modulator indicating an NPC1L1 agonist. Preferred methods in accordance herewith further comprise reducing or depleting cholesterol from the plasma membrane of the cells prior to step (a).

In all assays disclosed herein, the experiments may be performed with a control experiment lacking or minimally possessing any NPC1L1-binding. The control experiment may be performed, for example with a cell or cell membrane lacking any functional NPC1L1 including but not limited to a different cell line not expressing NPC1L1, cells from which genomic NPC1L1 DNA has been disrupted or deleted, or cells where endogenous NPC1L1 RNA has been depleted, for example, by RNAi. When the control experiment is performed, the level of binding observed in the presence of candidate NPC1L1 being tested for the presence of an antagonist can be compared with that observed in the control experiment.

### Cholesterol Reduction/Depletion Assays

Discovery of a robust endogenous NPC1L1-like activity in MDCK cells provided a means to assess, physiologically, what results after perturbing cholesterol homeostasis by either depleting cholesterol from the plasma membrane (e.g., by using methyl-β-cyclodextrin (“MβCD”)) and/or blocking endogenous cholesterol synthesis (e.g., with the HMG CoA reductase inhibitor lovastatin). Interestingly, and in agreement with a recent report indicating that the HMG CoA reductase inhibitor mevinolin up-regulates transcription of NPC1L1 in CaCo-2 cells (Alrefai et al., 2007 Am. J. Physiol. Gastrointest. Liver Physiol. 292(1):G369-376), serum-depleted MDCK cells respond to inhibition of HMG CoA reductase by increasing the amount of NPC1L1 expressed at the cell surface. Notably, this mechanism was readily apparent only in cells grown in lipoprotein depleted media, suggesting that, under normal conditions, the acquisition of lipoproteins, cholesterol ester and cholesterol through LDLR may bypass the need for up-regulating surface NPC1L1 levels. These observations support the contention that NPC1L1 may act as part of a cholesterol transport mechanism in MDCKII cells.

The determination of whether MDCK cells, although sensing and responding to variations in endogenous cholesterol, could actually transport enough cholesterol, in an NPC1L1-dependent manner was an important one. Using an assay similar to that reported for monitoring EZE-sensitive cholesterol influx into McArdles RH7777 rat hepatoma cells overexpressing human NPC1L1 tagged with GFP (Yu et al., 2006 J. Biol. Chem. 281:6616-6624), after overexpressing NPC1L1 in the apical membrane of MDCKII cells and depleting the membrane with βmCD, cholesterol flux was significantly sensitive to EZE (Yu et al., 2006 Biol. Chem. 281:6616-6624).

Accordingly, in specific embodiments, the present invention provides a method for identifying an NPC1L1 modulator capable of effecting NPC1L1-mediated cholesterol absorption or flux, which comprises: (a) providing MDCK cells overexpressing NPC1L1; (b) reducing or depleting cholesterol from the plasma membrane (e.g., by using methyl-β-cyclodextrin or through any suitable alternative means); (c) contacting the MDCK cells with detectably labeled sterol (e.g., cholesterol) or 5α-stanol; (d) providing a candidate NPC1L1 modulator to the MDCK cells; and (e) measuring uptake or influx of the detectably labeled sterol or 5α-stanol; a decrease in cholesterol influx upon the addition of the candidate NPC1L1 modulator indicating an NPC1L1 antagonist; and an increase in cholesterol influx indicating an NPC1L1 agonist. In specific embodiments, the MDCK cells are transfected with nucleic acid encoding either dog or human NPC1L1. In specific embodiments, the cells are incubated with methyl-β-cyclodextrin or suitable agent for a sufficient period of time to allow for significant depletion of cholesterol from the plasma membrane. In specific embodiments, a cellular lysate is prepared between steps (d) and (e). In specific embodiments, detection of uptake of the detectably labeled sterol or 5α-stanol is measured by liquid scintillation counting of a cellular lysate. In additional embodiments, the method further comprises the administration of a known NPC1L1 modulator as a comparator or control. In the situations where a known NPC1L1 antagonist is present, a decrease in cholesterol influx as compared to the control without the candidate NPC1L1 modulator indicates an NPC1L1 antagonist. Similarly, where the control is in the absence of an NPC1L1 antagonist, a decrease in cholesterol influx as compared to the control without the candidate NPC1L1 modulator indicates an NPC1L1 antagonist.

In additional embodiments, the present invention provides a method for identifying an NPC1L1 modulator capable of effecting NPC1L1-mediated cholesterol absorption or flux, which comprises: (a) providing MDCK cells overexpressing NPC1L1; (b) inhibiting or blocking endogenous cholesterol synthesis (e.g., with the HMG CoA reductase inhibitor lovastatin or by any suitable alternative means); (c) contacting the MDCK cells with detectably labeled sterol (e.g., cholesterol) or 5α-stanol; (d) providing a candidate NPC1L1 modulator to the MDCK cells; and (e) measuring uptake or influx of the detectably labeled sterol or 5α-stanol; a decrease in cholesterol influx upon the addition of the candidate NPC1L1 modulator indicating an NPC1L1 antagonist; and an increase in cholesterol influx indicating an NPC1L1 agonist. In specific embodiments, the MDCK cells are transfected with nucleic acid encoding human or dog NPC1L1. In specific embodiments, the cells are incubated with methyl-β-cyclodextrin or suitable agent for a sufficient period of time to allow for significant depletion of cholesterol from the plasma membrane. In specific embodiments, a cellular lysate is prepared between steps (d) and (e). In specific embodiments, detection of uptake of the detectably labeled sterol or 5α-stanol is measured by liquid scintillation counting of a cellular lysate. In additional embodiments, the method further comprises the administration of a known NPC1L1 modulator as a comparator or control. In the situations where a known NPC1L1 antagonist is present, a decrease in cholesterol influx as compared to the control without the candidate NPC1L1 modulator indicates an NPC1L1 antagonist. Similarly, where the control is in the absence of an NPC1L1 antagonist, a decrease in cholesterol influx as compared to the control without the candidate NPC1L1 modulator indicates an NPC1L1 antagonist.

### Cells of Use in the Disclosed Assays

MDCK cells of use in the assays disclosed herein may be any MDCK cells or MDCK-derived cells including but not limited to that described in Blacarova-Stander et al., 1984 EMBO J. 3:2687-2694; Louvard, 1980 Proc. Natl. Acad. Set USA 77(7): 4132-4136; Cohen & Miisch, 2003 Methods 30:269-276, or as deposited as ATCC Number CCL-34. In preferred embodiments, the MDCK cells employed in the disclosed assays are those MDCK cells characterized as MDCKII cells, see, e.g., Reinsch & Karsenti, 1994 J. Cell Biol. 126(6):1509-1526 (“MDCKII” cells).

In preferred embodiments, the MDCK cells are polarized. Cells fully polarize after roughly 2-3 days on plates. This allows for high expression of endogenous NPC1L1 .

In preferred embodiments, the MDCK cells express greater than 1,500,000 ligand binding sites of NPC1L1 on the cell surface. This may be measured and the appropriate concentration of ligand binding sites determined using available methods routinely employed by the skilled artisan and as described herein for the binding assays.

In specific embodiments, the cells may be manipulated to overexpress NPC1L1 by any method available to the skilled artisan, including but not limited to induction of NPC1L1 expression, induction of increased NPC1L1 available at the cell surface, or transient transfection of the cells with nucleic acid encoding NPC1L1 protein.

In specific embodiments, a nucleic acid encoding an NPC1L1 polypeptide is transfected into an MDCK cell, and the NPC1L1 expressed is incorporated into the membrane of the cell, as described, for instance, in Yu et al., 2006 J. Biol. Chem. 281 (10): 6616-6624. Stable transfection of MDCK cells with human NPC1L1 led to a 10-20 fold increase in [³H]AS binding compared to the MDCK background tested. Dog or human NPC1L1 were over-expressed in MDCKII cells to increase the amount of NPC1L1-mediated cholesterol influx relative to non-specific delivery of cholesterol. Such an approach, in a similar manner to the over-expression of NPC1L1 in CaCo-2 cells (Yamanashi et al., 2007 J. Pharmacol. Exp. Ther. 320(2):559-564), allowed the delivery of [³H]cholesterol or [³H]sitosterol to MDCKII cells in an EZE-sensitive manner and with a pharmacology that resembled that of the [³H]AS binding assay, supporting its utility for identifying novel inhibitors of NPC1L1-mediated processes.

Membrane preparations bearing NPC1L1 are also of use in the binding assays disclosed herein. A membrane fraction may be isolated from MDCK cells and used as a source of NPC1L1 for assay. Similar to above, preferably the membrane is derived from a cell expressing greater than 1,500,000 binding sites for NPC1L1/cell. Membrane preparations may be obtained according to methods fully available to the skilled artisan, see, e.g., Yu et al., 2006 J. Biol. Chem. 281(10):6616-6624. The membrane preparation may be in vesicular or non-vesicular form.

Alternatively, the disclosed binding assays may be run with cell lysates prepared from MDCK cells. Similar to above, preferably the membrane is derived from a cell expressing greater than 1,500,000 binding sites for NPC1L1 per cell. Cellular lysates may be obtained according to conventional methods in the art.

### NPC1L1 of Use in the Disclosed Assays

NPC1L1 useful in the assays disclosed herein is a protein or fragment thereof characterized by:

(a) one or more of the following characteristics: (i) its homology (>80%) on an amino acid level to previously characterized NPC1L1 proteins; and (ii) the ability of encoding nucleic acid to hybridize to the complement of nucleic acid encoding known NPC1L1 proteins (i.e., a protein confirmed to be NPC1L1 based on binding to known NPC1L1 ligands (e.g., sterol, 5α-stanol, EZE or its derivatives) or the ability to mediate cholesterol influx into suitable cells (including but not limited to HepG2, cells, CaCo-2 cells and MDCK cells (inclusive of MDCKII cells)); and

(b) one or more of the following characteristics: (i) the ability of the candidate NPC1L1 to bind known NPC1L1 ligands (e.g., EZE or its derivatives, including but not limited to substituted azetidinones, substituted 2-azetidinones, substituted 2-azetidinone-glucuronide, and ezetimibe-glucuronide), and (ii) the ability to mediate cholesterol influx into suitable cells, including but not limited to HepG2 cells, CaCo-2 cells and MDCK cells (inclusive of MDCKII cells over-expressing NPC1L1)).

A fragment of use in the disclosed assays should be capable of binding at least one previously characterized NPC1L1 modulator, including but not limited to sterol, 5α-stanol, EZE and its derivatives and/or possess the ability to induce cholesterol influx into suitable cells, including but not limited to HepG2 cells, CaCo-2 cells and MDCK cells (including but not limited to MDCKII cells).

In specific embodiments, the NPC1L1 used in the disclosed assays is at least about 70% identical, preferably at least about 80% identical, more preferably at least about 90% identical and most preferably at least about 95% identical (e.g., 95%, 96%, 97%, 98%, 99%, 100%) on the amino acid level to a previously characterized NPC1L1 protein when the comparison is performed by a BLAST algorithm; the parameters of the algorithm being selected to give the largest match between the respective sequences over the entire length of the respective reference sequences. BLAST algorithms are known in the art; see, e.g., Altschul, S. F., et al., (1990) J. Mol. Biol. 215: 403-410; Gish, W., et al., (1993) Nature Genet. 3: 266-272; Madden, T. L., et al., (1996) Meth. Enzymol. 266: 131-141; Altschul, S. F., et al., (1997) Nucleic Acids Res. 25: 3389-3402; Zhang, J., et al., (1997) Genome Res. 7: 649-656; Wootton, J. C., et al., (1993) Comput. Chem. 17: 149-163; Hancock, J. M., et al., (1994) Comput. Appl. Biosci. 10: 67-70.

Alternatively, a functional equivalent of NPC1L1 may be employed in the disclosed assays. Functional equivalents of NPC1L1 include but are not limited to isoforms and variants of previously characterized NPC1L1 protein, and derivatives of previously characterized NPC1L1 protein, including but not limited to post-translationally-modified and chemically-modified derivatives of NPC1L1, fragments of previously characterized NPC1L1 or any of the foregoing. Functional equivalents also contemplates function-conserved variants, defined herein as those sequences or proteins in which one or more amino acid residues in a previously characterized NPC1L1 have been changed without altering the overall conformation and function. The changes in such function-conserved variants include, but are by no means limited to, replacement of an amino acid with one having similar properties. Such conservative amino acid substitutions, as one of ordinary skill in the art will appreciate, are substitutions that replace an amino acid residue with one imparting similar or better (for the intended purpose) functional and/or chemical characteristics. For example, conservative amino acid substitutions are often ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). The purpose for making a substitution is not significant and can include, but is by no means limited to, replacing a residue with one better able to maintain or enhance the structure of the molecule, the charge or hydrophobicity of the molecule, or the size of the molecule. For instance, one may desire simply to substitute a less desired residue with one of the same polarity or charge. Such modifications can be introduced by standard techniques known in the art, such as site-directed mutagenesis and PCR-mediated mutagenesis.

Functional equivalents should exhibit at least 10% and in order of increasing preference, 20%, 30%, 40%, 50%, 60%, 70,%, 80%, 90%, or 95% of: (i) the degree of binding to NPC1L1 or cell, membrane preparation or cell lysate expressing greater than 1,500,000 binding sites for NPC1L1 that known NPC1L1 modulators (e.g., EZE, its derivatives, including but not limited to substituted azetidinones, substituted 2-azetidinones, substituted 2-azetidinone-glucuronide, and ezetimibe-glucuronide) exhibit; or (ii) the degree of cholesterol influx mediated by known NPC1L1 modulators in a given assay. In specific embodiments, the activity of (ii) is the absorption of cholesterol in an EZE-sensitive manner (i.e., where the absorption of cholesterol is significantly reduced by the act of providing EZE or its derivatives).

The NPC1L1 expressed may be derived from any species. In specific embodiments, the NPC1L1 employed is derived from a dog (see, e.g., GenBank Accession Nos. NP_{—}001091019, ABK32534), with particular encoding nucleic acid disclosed in DQ897676. In preferred embodiments, the dog NPC1L1 is that disclosed in SEQ ID NO: 5 (an encoding nucleic acid provided in SEQ ID NO: 4). In other embodiments, the NPC1L1 employed is derived from a human (see, e.g., GenBank Accession Nos. AA17179, NP_{—}037521, AAF20397, AAF20396, AAR97886, EAL23753, AF192522; (see, Davies, et al., (2000) Genomics 65(2): 137-45), SEQ ID NO: 4 of International Publication No. WO 2005/062824 A2). In further embodiments, the NPC1L1 employed is derived from a mouse (see, e.g., GenBank Accession Nos. AAI31789, AAI31790, NP_{—}997125, EDL40576, AAR97887, CAI24395, SEQ ID NO: 12 of International Publication No. WO 2005/062824 A2). In additional embodiments, the NPC1L1 employed is derived from a rat (see, e.g., GenBank Accession Nos. NP_{—}001002025, AAR97888, SEQ ID NO: 2 of International Publication No. WO 2005/062824 A2). In alternative embodiments, the NPC1L1 employed is derived from a macaque (see, e.g., GenBank Accession No. ABK32536, ABK32535, NP_{—}001071157).

In specific embodiments, the NPC1L1 is encoded by nucleic acid which hybridizes to the complement of nucleic acid encoding a previously characterized NPC1L1. Preferably, the nucleic acids hybridize under low stringency conditions, more preferably under moderate stringency conditions and most preferably under high stringency conditions. Methods for hybridizing nucleic acids are well-known in the art; see, e.g., Ausubel, Current Protocols in Molecular Biology, John Wiley & Sons, N.Y., 6.3.1-6.3.6, 1989. For purposes of exemplification and not limitation, low stringency conditions may, in specific embodiments, use the following conditions: (i) 55° C., 5× sodium chloride/sodium citrate (“SSC”), 0.1% SDS, 0.25% milk, and no formamide at 42° C.; or (ii) 30% formamide, 5×SSC, 0.5% SDS at 42° C. For purposes of exemplification and not limitation, moderately stringent hybridization conditions may, in specific embodiments, use the foregoing conditions with some modifications, e.g., hybridization in 40% formamide, with 5× (or 6×) SSC. One specific example of moderately stringent hybridization conditions is the following protocol: a prewashing solution containing 5× sodium chloride/sodium citrate (SSC), 0.5% w/v SDS, 1.0 mM EDTA (pH 8.0), hybridization buffer of about 50% v/v formamide, 6×SSC, and a hybridization temperature of 55° C. (or other similar hybridization solutions, such as one containing about 50% v/v formamide, with a hybridization temperature of 42° C.), and washing conditions of 60° C., in 0.5×SSC, 0.1% w/v SDS. For purposes of exemplification and not limitation, stringent hybridization conditions may, in specific embodiments, use the conditions for low stringency with some modifications, e.g., hybridization in 50% formamide, with 5× (or 6×) SSC and possibly at a higher temperature (e.g., higher than 42° C.). One specific example of high stringency hybridization conditions is the following: 6×SSC at 45° C., followed by one or more washes in 0.1×SSC, 0.2% SDS at 68° C. One of skill in the art may, furthermore, manipulate the hybridization and/or washing conditions to increase or decrease the stringency of hybridization such that nucleic acids comprising nucleotide sequences that are, for example, at least 80, 85, 90, 95, 98, or 99% identical to each other typically remain hybridized to each other. The basic parameters affecting the choice of hybridization conditions and guidance for devising suitable conditions are set forth by Sambrook et al., Molecular Cloning: A Laboratory Manual, Cold Spring Harbor Laboratory Press, Cold Spring Harbor, N.Y., chapters 9 and 11, 1989 and Ausubel et al. (eds), Current Protocols in Molecular Biology, John Wiley & Sons, Inc., sections 2.10 and 6.3-6.4, 1995. Such parameters can be readily determined by those having ordinary skill in the art based on, for example, the length and/or base composition of the DNA.

NPC1L1 Obtained from MDCH Cells

The present invention relates to isolated or purified canine NPC1L1 polypeptide wherein said polypeptide comprises SEQ ID NO: 5.

The proteins, polypeptides and antigenic fragments of this invention may be purified by standard methods, including, but not limited to, salt or alcohol precipitation, affinity chromatography (e.g., used in conjunction with a purification tagged NPC1L1 polypeptide as discussed above), preparative disc-gel electrophoresis, isoelectric focusing, high pressure liquid chromatography (HPLC), reversed-phase HPLC, gel filtration, cation and anion exchange and partition chromatography, and countercurrent distribution. Such purification methods are well known in the art and are disclosed, e.g., in “Guide to Protein Purification”, Methods in Enzymology, Vol. 182, M. Deutscher, Ed., 1990, Academic Press, New York, N.Y.

Particularly where an NPC1L1 polypeptide is being isolated from a cellular or tissue source, it is preferable to include one or more inhibitors of proteolytic enzymes in the assay system, such as phenylmethanesulfonyl fluoride (PMSF), Pefabloc SC, pepstatin, leupeptin, chymostatin and EDTA.

Polypeptides disclosed herein may additionally be produced by chemical synthesis or by the application of recombinant DNA technology. Any method available to the skilled artisan may be utilized including, but not limited to, through direct synthesis or via various recombinant expression techniques available (for instance, in yeast, E. coli, or any other suitable expression system). In specific embodiments, the polypeptide of the invention may be prepared by culturing transformed host cells under culture conditions suitable to express the recombinant polypeptide. The resulting expressed polypeptide may then be purified from such culture (i.e., from culture medium or cell extracts) using known purification processes including, but not limited to, gel filtration and ion exchange chromatography. Purified, recombinant polypeptides form specific embodiments of the present invention. The polypeptide thus purified is substantially free of other mammalian polypeptides other than those polypeptides affirmatively adjoined or added after or during purification and is defined in accordance with the present invention as an “isolated polypeptide” or “recombinant polypeptide”; such isolated or recombinant polypeptides of the invention include polypeptides of the invention, fragments, and variants.

The present invention also relates to isolated nucleic acid encoding dog NPC1L1 polypeptide which comprises SEQ ID NO: 5. In particular embodiments, the isolated nucleic acid comprises SEQ ID NO: 4.

Nucleic acid encoding the disclosed polypeptides may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like.

In specific embodiments, the heterologous promoter is recognized by a eukaryotic RNA polymerase. One example of a promoter suitable for use in the present invention is the immediate early human cytomegalovirus promoter (Chapman et al., 1991 Nucl. Acids Res. 19:3979-3986). Further examples of promoters that can be used in the present invention are the cytomegalovirus (CMV) promoter (see, e.g., U.S. Pat. Nos. 5,385,839 and 5,168,062), the SV40 early promoter region (see, e.g., Benoist, et al., (1981) Nature 290: 304-310), the promoter contained in the 3′ long terminal repeat of Rous sarcoma virus (see, e.g., Yamamoto, et al., (1980) Cell 22: 787-797), the herpes thymidine kinase promoter (see, e.g., Wagner, et al., (1981) Proc. Natl. Acad. Sci. USA 78: 1441-1445), the regulatory sequences of the metallothionein gene (see, e.g., Brinster, et al., (1982) Nature 296: 39-42); prokaryotic expression vectors such as the β-lactamase promoter (see, e.g., VIIIa-Komaroff, et al., (1978) Proc. Natl. Acad. Sci. USA 75: 3727-3731), or the tac promoter (see, e.g., DeBoer, et al., (1983) Proc. Natl. Acad. Sci. USA 80: 21-25); see also “Useful proteins from recombinant bacteria” in Scientific American (1980) 242: 74-94; and promoter elements from yeast or other fungi such as the Gal 4 promoter, the ADC (alcohol dehydrogenase) promoter, PGK (phosphoglycerol kinase) promoter or the alkaline phosphatase promoter; albeit those of skill in the art can appreciate that any promoter capable of effecting expression of the heterologous nucleic acid in the intended host can be used in accordance with the methods of the present invention. The promoter may comprise a regulatable sequence such as the Tet operator sequence. Sequences such as these that offer the potential for regulation of transcription and expression are useful in circumstances where repression/modulation of gene transcription is sought.

Nucleic acid as referred to herein may be DNA and/or RNA, and may be double or single stranded. The nucleic acid may be in the form of an expression cassette. In this respect, specific embodiments of the present invention relate to a gene expression cassette comprising (a) nucleic acid encoding SEQ ID NO: 5 (or nucleic acid comprising SEQ ID NO: 4); (b) a heterologous promoter operatively linked to the nucleic acid; and (c) a transcription termination signal.

The present invention also encompasses vectors comprising the described nucleic acid encoding SEQ ID NO: 5 (or nucleic acid comprising SEQ ID NO: 4). Known recombinant nucleic acid methodology may be used to incorporate the nucleic acid sequences into various vector constructs.

Vectors that can be used in this invention include plasmids, viruses, bacteriophage, integratable DNA fragments, and other vehicles that may facilitate introduction of the nucleic acids into the genome of the host. Plasmids are the most commonly used form of vector but all other forms of vectors which serve a similar function and which are, or become, known in the art are suitable for use herein. See, e.g., Pouwels, et al., Cloning Vectors: A Laboratory Manual, 1985 and Supplements, Elsevier, N.Y., and Rodriguez et al. (eds.), Vectors: A Survey of Molecular Cloning Vectors and Their Uses, 1988, Buttersworth, Boston, Mass.

The term “expression system” means a host cell and compatible vector which, under suitable conditions, can express a protein or nucleic acid which is carried by the vector and introduced to the host cell. Common expression systems include E. coli host cells and plasmid vectors, insect host cells and Baculovirus vectors, and mammalian host cells and vectors.

Expression of nucleic acids encoding the NPC1L1 polypeptides of this invention can be carried out by conventional methods in either prokaryotic or eukaryotic cells. Although E. coli host cells are employed most frequently in prokaryotic systems, many other bacteria, such as various strains of Pseudomonas and Bacillus, are known in the art and can be used as well. Suitable host cells for expressing nucleic acids encoding the NPC1L1 polypeptides include prokaryotes and higher eukaryotes. Prokaryotes include both gram-negative and gram-positive organisms, e.g., E. coli and B. subtilis. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. A representative vector for amplifying DNA is pBR322 or many of its derivatives (e.g., pUC18 or 19). Vectors that can be used to express the NPC1L1 polypeptides include, but are not limited to, those containing the lac promoter (pUC-series); tip promoter (pBR322-tip); Ipp promoter (the pIN-series); lambda-pP or pR promoters (pOTS); or hybrid promoters such as ptac (pDR540). See Brosius et al., “Expression Vectors Employing Lambda-, trp-, lac-, and Ipp-derived Promoters”, in Rodriguez and Denhardt (eds.) Vectors: A Survey of Molecular Cloning Vectors and Their Uses, 1988, Buttersworth, Boston, pp. 205-236. Many polypeptides can be expressed, at high levels, in an E. coli/T7 expression system as disclosed in U.S. Pat. Nos. 4,952,496; 5,693,489 and 5,869,320 and in Davanloo, P., et al., (1984) Proc. Natl. Acad. Sci. USA 81: 2035-2039; Studier, F. W., et al., (1986) J. Mol. Biol. 189: 113-130; Rosenberg, A. H., et al., (1987) Gene 56: 125-135; and Dunn, J. J., et al., (1988) Gene 68: 259.

Higher eukaryotic tissue culture cells may also be used for the recombinant production of the NPC1L1 polypeptides of the invention. Although any higher eukaryotic tissue culture cell line might be used, including insect baculovirus expression systems, mammalian cells are preferred. Transformation or transfection and propagation of such cells have become a routine procedure. Examples of useful cell lines include HeLa cells, chinese hamster ovary (CHO) cell lines, J774 cells, Caco2 cells, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also, usually, contain a selection gene or amplification gene. Suitable expression vectors may be plasmids, viruses, or retroviruses carrying promoters derived, e.g., from such sources as adenovirus, SV40, parvoviruses, vaccinia virus, or cytomegalovirus. Examples of expression vectors include pCR®3.1, pcDNA1, pCD (Okayama, et al., (1985) Mol. Cell. Biol. 5: 1136), pMClneo Poly-A (Thomas, et al., (1987) Cell 51: 503), pREP8, pSVSPORT and derivatives thereof, and baculovirus vectors such as pAC373 or pAC610.

The present invention also includes fusions which include of the disclosed NPC1L1 polypeptides (polypeptides comprising SEQ ID NO: 5) and NPC1L1 polynucleotides of the present invention (nucleic acid encoding SEQ ID NO: 5 or comprising SEQ ID NO: 4) and a second polypeptide or polynucleotide moiety, which may be referred to as a “tag”. The fused polypeptides of the invention may be conveniently constructed, for example, by insertion of a polynucleotide of the invention or fragment thereof into an expression vector. The fusions of the invention may include tags which facilitate purification or detection. Such tags include glutathione-S-transferase (GST), hexahistidine (His6) tags, maltose binding protein (MBP) tags, haemagglutinin (HA) tags, cellulose binding protein (CBP) tags and myc tags. Detectable tags such as ³²P, ³⁵S, ³H, ^{99m}Tc, ¹²³I, ¹¹¹In, ⁶⁸Ga, ¹⁸F, ¹²⁵I, ^{113m}In, ⁷⁶Br, ⁶⁷Ga, ^{99m}Tc, ¹²³I, ¹¹¹In and ⁶⁸Ga may also be used to label the polypeptides and polynucleotides of the invention. Methods for constructing and using such fusions are very conventional and well known in the art. Modifications (e.g., post-translational modifications) that occur in a polypeptide often will be a function of how it is made. For polypeptides made by expressing a cloned gene in a host, for instance, the nature and extent of the modifications, in large part, will be determined by the host cell's post-translational modification capacity and the modification signals present in the polypeptide amino acid sequence. For instance, as is well known, glycosylation often does not occur in bacterial hosts such as E. coli. Accordingly, when glycosylation is desired, a polypeptide can be expressed in a glycosylating host, generally a eukaryotic cell. Insect cells often carry out post-translational glycosylations which are similar to those of mammalian cells.

For this reason, insect cell expression systems have been developed to express, efficiently, mammalian proteins having native patterns of glycosylation. An insect cell which may be used in this invention is any cell derived from an organism of the class Insecta. Preferably, the insect is Spodoptera frugiperda (Sf9 or 5121) or Trichoplusia ni (High 5). Examples of insect expression systems that can be used with the present invention, for example to produce NPC1L1 polypeptide, include Bac-To-Bac (Invitrogen Corporation, Carlsbad, Calif.) or Gateway (Invitrogen Corporation, Carlsbad, Calif.). If desired, deglycosylation enzymes can be used to remove carbohydrates attached during production in eukaryotic expression systems.

Other modifications may also include addition of aliphatic esters or amides to the polypeptide carboxyl terminus. The present invention also includes analogs of the NPC1L1 polypeptides which contain modifications, such as incorporation of unnatural amino acid residues, or phosphorylated amino acid residues such as phosphotyrosine, phosphoserine or phosphothreonine residues. Other potential modifications include sulfonation, biotinylation, or the addition of other moieties. For example, the NPC1L1 polypeptides of the invention may be appended with a polymer which increases the half-life of the peptide in the body of a subject. Preferred polymers include polyethylene glycol (PEG) (e.g., PEG with a molecular weight of 2 kDa, 5 kDa, 10 kDa, 12 kDa, 20 kDa, 30 kDa and 40 kDa), dextran and monomethoxypolyethylene glycol (mPEG).

The peptides of the invention may also be cyclized. Specifically, the amino- and carboxy-terminal residues of an NPC1L1 polypeptide or two internal residues of an NPC1L1 polypeptide of the invention can be fused to create a cyclized peptide. Methods for cyclizing peptides are conventional and very well known in the art; for example, see Gurrath, et al., (1992) Eur. J. Biochem. 210: 911-921.

The present invention further encompasses, as particular embodiments hereof, cells, isolated populations of cells, membrane fractions thereof, and non-human transgenic animals comprising the nucleic acid and vectors described herein. In particular aspect, the present invention encompasses MDCK cells and membrane fractions thereof expressing recombinant (i.e., derived by man) NPC1L1 protein including but not limited to that of SEQ ID NO: 5. Said NPC1L1 protein may be any NPC1L1 protein described herein and includes but is by no means limited to that comprising SEQ ID NO: 5. “Recombinant” NPC1L1 includes but is not limited to NPC1L1 expressed as a result of transfection of nucleic acid encoding NPC1L1 into MDCK cells, and NPC1L1 expressed through the acts of incorporating and activating a promoter operably linked to nucleic acid encoding NPC1L1 (or alternatively, activating a native promoter operably linked to nucleic acid encoding NPC1L1) such that NPC1L1 is overexpressed. A coding sequence is “under the control of”, “functionally associated with”, “operably linked to” or “operably associated with” transcriptional and translational control sequences in a cell when the sequences direct RNA polymerase mediated transcription of the coding sequence into RNA, preferably mRNA, which then may be NRA spliced (if it contains introns) and, optionally, translated into a protein encoded by the coding sequence.

The following non-limiting examples are presented to better illustrate the workings of the invention.

### Example 1

### Materials

Restriction enzymes and Pfusion polymerase were from New England Biolabs (Beverly, Mass.). pcDNA5-FRT-TOPO, pcDNA5-FRT, Superscriptli and STBL2 competent cells were purchased from Invitrogen (Carlsbad, Calif.). Synthetic oligonucleotides were synthesized by IDT (Coralville, Iowa). Tri Reagent for RNA preparation was obtained from Molecular Research Center (Cinncinati, Ohio). dNIP's were purchased from Roche Diagnostics, (Indianapolis, Ind.), RNeasy columns from Qiagen® (Valencia, Calif.), and Chromaspin columns from Clontech (Mountain View, Calif.). Dye terminator sequence reactions were performed with the ABI Big Dye 3.1 sequencing kit and analyzed with an ABI3100 genetic analyzer, both from Applied Biosystems (Foster City, Calif.). Human embryonic kidney (HEK) 293 cells, HepG2, LLC-PKI and CaCo-2 cell lines were from American Type Culture Collection (Manassas, Va.). MDCKII cells (see, Louvard 1980 Proc. Natl. Acad. Sci. USA 77:4132-4136) and TsA-201 cells (see, Hanner et al., 2001 Biochemistry 40:11687-11697) were provided. Fugene6 transfection reagent was obtained from Roche (Indianapolis, Ind.). Generation and maintenance of a stable cell line expressing rat NPC1L1 in HEK 293 cells (rNPC1L1/HEK293) (see, Garcia-Calvo et al., 2005 Proc. Natl. Acad. Sci. USA 102:8132-8137), and procedures for handling TsA-201 cells and their transfection with FuGENE6 have been previously described; see, Hanner et al., 2001 Biochemistry 40:11687-11697. LLC-PKI cells were maintained in medium 199+Glutamax, CaCo-2 and MDCKII cells in DMEM+Glutamax (Sigma) and HepG2 cells in Eagles minimum essential medium. All media were supplemented with 10% FBS, penicillin and streptomycin and cells were grown at 37° C. in 5% CO2. Ezetimibe (EZE), ezetimibe glucuronide (EZE-gluc) and the EZE-gluc-enantiomer (ent-1) were prepared as previously described; see Garcia-Calvo et al., supra. The propargyl sulphonamide, 4-[(2S,3R)-3-[(3S)-3-(4-fluorophenyl)-3-hydroxypropyl]-1-(4-{3-[(methylsulfonyl)amino]prop-1-yn-1-yl}phenyl)-4-oxoazetidin-2-yl]phenyl methyl-β-D-glucopyranosiduronate (PS) and the alkyl sulphonamide, 4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl β-D-gluteopyranosiduronic acid (AS) are described in Goulet et al., International Publication No. WO 2005/062824 A2. All other reagents were obtained from commercial sources and were of the highest purity commercially available.

### Example 2

### Preparation of [³H]AS

A solution of AS (2 mg, 0.0028 mmol) in 0.8 mL of anhydrous N,N-dimethylformamide was de-gassed at dry ice/acetone temperature in the presence of 5 mg 10% Pd/C (Sigma-Aldrich Chemical, 10% (dry basis) on activated carbon, wet, Degussa type). The mixture was stirred at 0° C. for 2 hr under 240 mmHg of carrier-free tritium gas (1.2 Ci, American Radiochemical Chemicals). Un-reacted tritium gas was removed, the catalyst was filtered through a syringe-less filter device (Whatman Autovial, 0.45 u PTFE), and the solvent and labile tritium were removed by concentration to near dryness. This procedure was repeated three times to ensure complete reduction of the C—C triple bonds and ensure high specific activity. The dried residue was re-suspended in 2 mL of ethanol and purified by HPLC (Phenomenex Luna Phenyl-Hexyl HPLC column, 9.4 mm×25 cm, CH₃CN:H₂O: TFA, 25:75:0.1 to 27:73:0.1 in 50 min). The [³H]AS eluted with a retention time of 32 min and was collected as a single fraction (210 mCi, 85 Ci/mmol, radiochemical purity ˜99% by HPLC). The identity was confirmed by LC/MS analysis and HPLC co-elution with unlabeled standard.

### Example 3

### Cell Based [³H]AS Binding

rNPC1L1/HEK293 and TsA201 cells were seeded at a density of 10,000 cells per well in 96-well poly-D-lysine coated plates and cells were allowed to attach for approximately 18 h at 37° C. TsA201 cells were subsequently transfected with dog NPC1L1/pcDNA5/FRT according to the manufacturer's instructions (Roche) and incubated for 3 days at 37° C. MDCKII-derived, LLC-PKI, HepG2, or CaCo-2 cells were seeded at a density of 25,000 cells per well in 96-well tissue culture treated plates, and cells were allowed to attach and differentiate for approximately 72 h at 37° C., except for CaCo-2 cells where differentiation took approximately 14 days at 37° C. For all binding studies, ˜5 nM [³H]AS in a total volume of ˜200 μl was added to the well, and cells were incubated under normal growth conditions for determined periods of time. Duplicate samples were averaged for each experimental point. For saturation binding experiments, cells were incubated with increasing concentrations of [³H]AS for 4 h. In competition binding experiments, cells were incubated with [³H]AS in the absence or presence of increasing concentrations of test compound. To determine the kinetics of ligand association, cells were incubated with [³H]AS for different periods of time. Dissociation kinetics were determined by addition of 100 μM EZE-gluc, and incubating for different periods of time. Nonspecific binding was defined in the presence of 100 μM EZE-gluc. At the end of the incubation period, cells were washed twice with 200 μl of pre-warmed DMEM to separate bound from free ligand, 1% SDS was added to the wells followed by 5 ml of Scintillant, and radioactivity associated with cells was determined using a β-counter. For acid wash experiments, cells were incubated with either 5 nM (rNPC1L1/HEK293) or 1 nM (MDCKII) [³H]AS for 2 h. Thereafter, plates were placed on ice and cells were washed twice with ice-cold PBS, followed by ice-cold acid wash with DMEM, pH 3.5, for 1, 5 or 15 minutes. Cells were then washed twice with PBS and re-incubated with [³H]AS for 2 h at 37° C. For Transwell experiments, MDCKII cells were seeded at 200,000 cells/well in a 24-well plate and incubated for 3 days at 37° C. [³H]AS was added to either the apical or basolateral compartment of the Transwell membrane, at a concentration of 1 nM, and incubation took place for 2 h at 37° C. Thereafter, both apical and basolateral compartments were washed three times with PBS, and the Transwell filter was cut out and its associated radioactivity determined using afi-counter. Data from saturation, competition and ligand dissociation experiments were analyzed as described in the literature; see, Priest, et al., 2004 Biochemistry 43:9866-9876; Knaus et al., 1995 Biochemistry 34:13627-13634. The association rate, k1, was determined by employing the pseudo-first-order rate equation k₁=k_{obs}([LR]ₑ/[L][LR]ₘₐₓ)) where [LR]ₘₐₓ is the concentration of the complex at equilibrium, [L] is the concentration of ligand, [LR]ₘₐₓ is the total receptor concentration, k_{obs} is the slope of the pseudo-first order plot, ln([LR]ₑ/([LR]ₑ−[LR]ₜ)) versus time, and [LR]ₜ is the receptor-ligand complex at one given time point t.

### Example 4

### Binding of [³H]AS TO RNPC1L1/HEK293 Cells

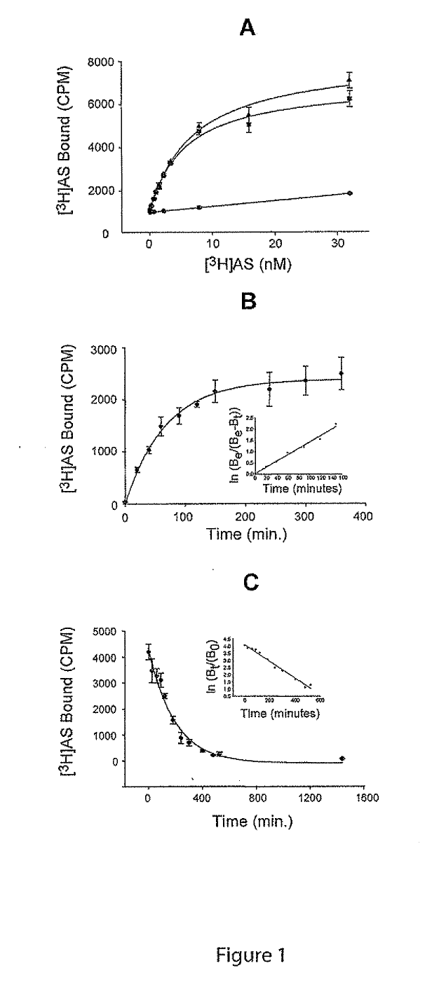

To identify cell lines that endogenously express NPC1L1 at the cell surface, a cell based assay that quantifies binding of the EZE analog, [³H]AS, to rat NPC1L1 heterologously expressed HEK293 cells (rNPC1L1/HEK293 cells) was established and validated. When rNPC1L1/HEK293 cells are incubated with increasing concentrations of [³H]AS, in the absence or presence of 100 μM Eze-Gluc, the radioligand associates specifically with cells as a saturatable function of ligand concentration and displays a good signal-noise ratio (FIG. 1A). As expected, the nonspecific binding component varied linearly with the [³H]AS concentration. A fit of the specific binding component to a single binding isotherm yielded an equilibrium dissociation constant, Kd, of 4.62±0.69 nM, and a maximum density of cell surface binding sites, Bmax, of 180 pM corresponding to 2.21×10⁶ binding sites/cell.

Incubation of rNPC1L1/HEK293 cells with 5 nM [311]AS results in a time-dependent association of ligand with cells that reaches equilibrium in ˜3 h (FIG. 1B). The nonspecific binding component is time-independent and has been subtracted from the experimental data. A semilogarithmic transformation of the data yielded a linear dependence (FIG. 1B, inset), as expected for a pseudo-first order reaction, and the slope of this line gives k_{obs} of 0.0208 min⁻¹. The association rate constant, k₁, calculated as described under Example 3, is 2.4×10⁶ M⁻¹ min⁻¹. Dissociation of cell bound [³H]AS, initiated by addition of 100 μM Eze-Gluc, followed a single mono-exponential decay with a t_{1/2} of ˜3 h, corresponding to L₁ of 0.0059 min⁻¹ (FIG. 1C). The K_{d} calculated from these rate constants was 2.46 nM, a value similar to that determined under equilibrium binding conditions (4.62 nM). These kinetic observations indicate that [³H]AS binds to a single class of sites through a simple bimolecular and fully reversible reaction.

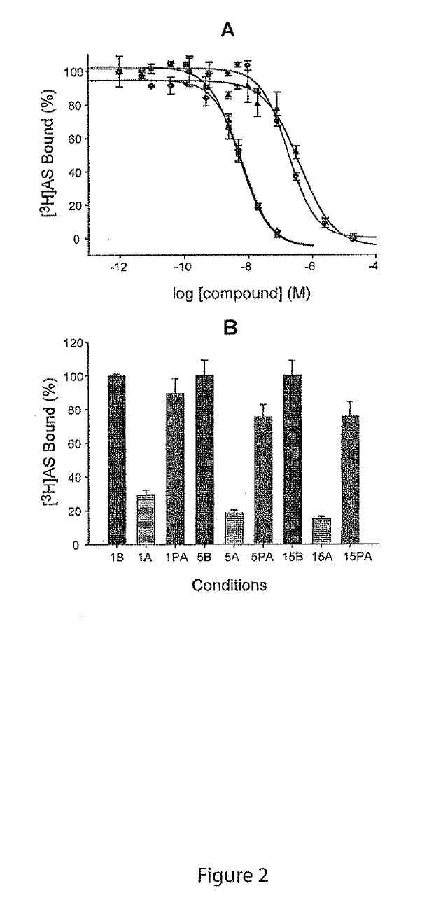

Binding of [³H]AS to rat NPC1L1/HEK293 cells was inhibited in a concentration dependent manner by increasing concentrations of AS, PS, EZE-gluc and EZE (FIG. 2A). Kᵢ values, determined as described above, are presented in Table 1 below and display the expected rank order of potency for interaction of these ligands with rat NPC1L1; Garcia-Calvo et al., 2005 Proc. Natl. Acad. Sci. USA 102:8132-8137.

To confirm that the non-covalent interaction between [³H]AS and rat NPC1L1 occurs at the cell surface, rNPC1L1/HEK293 cells were incubated with [³H]AS and subsequently acid washed with DMEM at pH 3.5. Such an approach has previously been previously used to characterize cell surface, non-covalent interactions; Hopkins & Trowbridge, 1983 J. Cell Biol. 97:508-521; Chen et al., 1998 Proc. Natl. Acad. USA 95:6373-6378. Treatment of cells at pH 3.5 for 1, 5 or 15 minutes led to dissociation of >70%, 80% and 85% of bound [³H]AS, respectively (FIG. 2B), indicating that the majority of radioligand binding sites are present at the cell surface and not at intracellular compartments. Importantly, after acid removal, incubation of cells with [³H]AS for 2 h at 37° C. causes re-binding of ligand at levels similar to those observed before the acid wash (FIG. 2B) indicating that the loss of radioligand after acid treatment was not due to any significant loss in cell viability. All data, taken together, strongly provide support for [³H]AS binding to cell surface expressed NPC1L1 and suggest that such a binding assay can be used to identify cell lines expressing this protein.

TABLE I
Binding properties of select β-lactams to rat NPC1L1/HEK293,
MDCKII or dog NPC1L1 expressed in TsA201 cells a, b.

| | | | Dog |
|---|---|---|---|
| | rNPC1L1/HEK293 | MDCKII | NPC1L1/TsA 201 |
| KD (AS) | 4.62 ± 0.69 nM | 0.59 ± 0.07 nM | 2.15 ± 0.39 nM |
| Ki (AS) | 2.35 ± 0.49 nM | 0.34 ± 0.04 nM | 1.00 ± 0.11 nM |
| Ki (PS) | 4.07 ± 1.47 nM | 0.33 ± 0.05 nM | 0.97 ± 0.08 nM |
| Ki (EZE) | 209 ± 40.4 nM | 14.01 ± 4.11 nM | 21.48 ± 7.56 nM |
| Ki (EZE- | 95.1 ± 8.62 nM | 3.51 ± 0.89 nM | 5.51 ± 1.52 nM |
| gluc) | | | |

a Kd values were determined from saturation experiments with increasing concentrations of [3H]AS. Values represent the mean ± SD of at least three independent determinations.
b Ki values were determined from competition experiments with [3H]AS and increasing concentrations of unlabeled β-lactams. Values represent the mean ± SD of 2-6 independent determinations.

### Example 5

### Identification of [³H]AS Binding Activity on the Apical Surface of Madin Darby Canine Kidney II (MDCKII) Cells

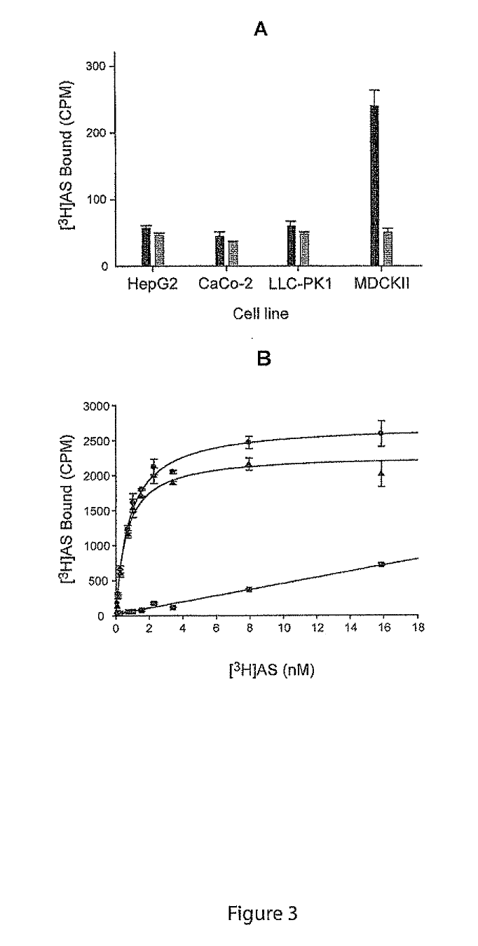

Based on the observation that [³H]AS binding to cells can accurately reflect the number of NPC1L1 molecules at the cell surface, HepG2, CaCo-2, LLC-PK1 or MDCKII cells were incubated with [³H]AS to determine whether any of these cell lines express NPC1L1. Notably, [³H]AS was only found to bind in a specific and robust manner to MDCKII cells (FIG. 3A). Saturation binding studies indeed indicate that [³H]AS binding to MDCKII cells occurs in a concentration dependent and saturable manner to a single class of sites that display a K_{d} of 0.59±0.07 nM and a Bᵣₙₐₓ of 87 pM, corresponding to 4.19×10⁵ sites/cell, (FIG. 3B). Under the growth and assay conditions described in Example 3 for CaCo-2, HepG2 and LLC-PKI cells, specific binding of [³H]AS was not observed in any case at ligand concentrations of up to 100 nM (data not shown).

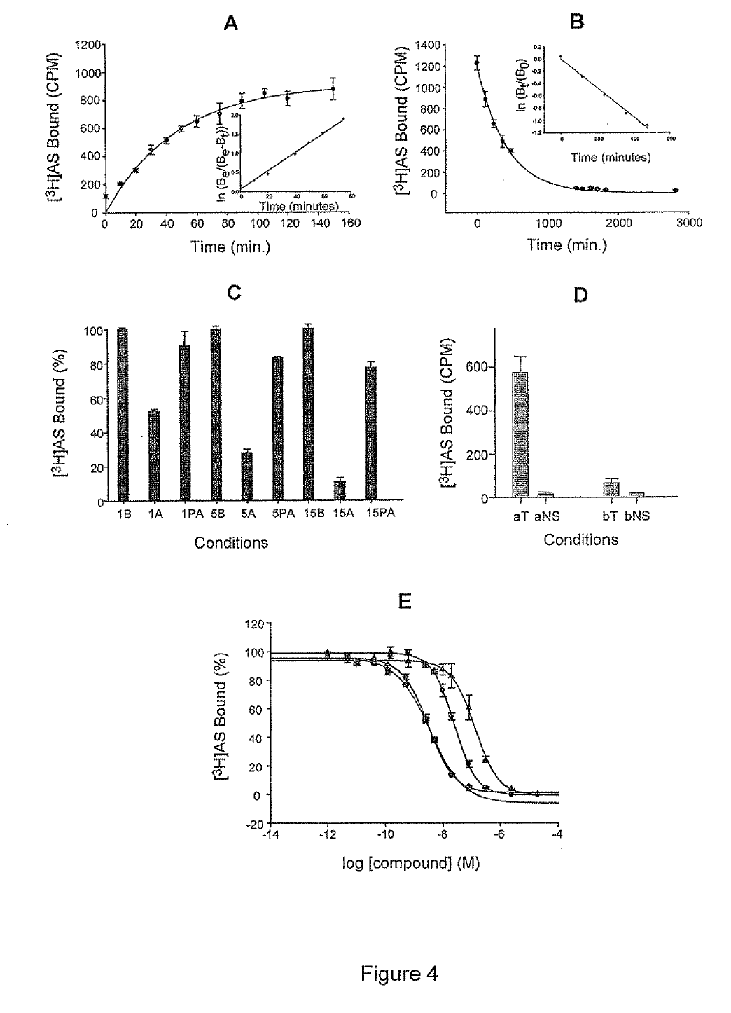

The kinetics of [³H]AS binding to MDCKII cells demonstrate that radioligand binding occurs through a simple bimolecular reaction. Thus, incubation of MDCKII cells with [³H]AS results in a time-dependent association of ligand with cells that reached equilibrium in 2 h. A semilogarithmic transformation of the data yielded a linear dependence, as expected for a pseudo-first order reaction, and the slope of this line gives a k_{obs} value of 0.0247 min⁻¹ (FIG. 4A) from which k₁ of 1,63×10⁷ M⁻¹min⁻¹ can be calculated. Dissociation of cell bound [³H]AS, initiated by addition of 100 μM Eze-Gluc, followed a single mono-exponential decay with a t_{1/2} of ˜3 h, corresponding to k₁ of 0.0023 min⁻¹ (FIG. 4B). The Kd calculated from these rate constants, 0.14 pM, is similar to that determined under equilibrium binding conditions, 0.59±0.07 nM, (FIG. 3B).

As previously observed with rNPC1L1/HEK293 cells, acid washing of MDCKII cells equilibrated with [³H]AS leads to dissociation of up to 85% of the radioligand (FIG. 4C). Likewise, after acid removal, [³H]AS binds to MDCKII cells at similar levels to those obtained before acid treatment indicating that the loss of binding was not due to any significant loss in cell viability, but to disruption of non-covalent interactions between ligand and cell surface expressed NPC1L1-like activity.

Since MDCKII cells, like enterocytes and hepatocytes, are polarized epithelial cells demonstrating microvilli and tight junctions, the distribution of [³H]AS binding sites was evaluated on Transwell supports where cells polarize to form an impermeable barrier between the apical and basolateral compartments. Addition of 1 nM [³H]AS to the apical side of the Transwell, which represents the apical surface of MDCKII cells, leads to significant specific [³H]AS binding (FIG. 4D). However, when the same amount of ligand is added to the basolateral side of the Transwell, corresponding to the basolateral surface of the MDCKII cells, specific [³H]AS binding is significantly lower than in the previous situation (FIG. 4D), indicating that most of the NPC1L1-like activity resides at the apical surface of MDCKII cells.

Furthermore, these results suggest that [³H]AS does not appreciably diffuse through the membrane, into the cell since in such a case it should be able to reach [³H]AS binding sites regardless of their apical or basolateral localization.

The NPC1L1-like activity expressed at the apical surface of MDCK cells was further characterized pharmacologically using a series of EZE-like compounds (FIG. 4E and Table I). Similarly to rat NPC1L1 expressed in HEK293 cells, AS and PS display equivalent potency as inhibitors of [³H]AS binding to MDCK cells, Kᵢ values of 0.34±0.04 nM (AS) and 0.33±0.05 nM (PS), respectively, with EZE-gluc being ˜10-fold weaker, Kᵢ of 3.51±0.89 nM, and EZE being the weakest of all tested analogs with a Kᵢ of 14.01±4.11 nM. It is worth noting that although the relative potencies of these compounds are similar for rat NPC1L1 expressed in HEK293 cells and MDCK cells, the absolute affinities are higher for MDCK cells.

### Example 6

### Cloning of Dog NPC1L1 and Expression in MDCKII Cells

Total RNA was isolated from 3×10⁷ MDCKII cells either 5- or 9-days post-splitting using TriReagente (Molecular Research Center, Cincinnatti, Ohio) and purified with RNeasy columns. Single stranded cDNA was synthesized from total RNA using Superscript™ II (Invitrogen, Carlsbad, Calif.) and random hexamer primers and subsequently purified with Chromaspin 200 following conditions suggested by the manufacturer (Clontech). BLAST searches of public DNA databases with the human NPC1L1 protein sequence identified a partial sequence for dog NPC1L1. Based on alignments with multiple sequences for NPC1L1 this dog sequence was missing its 3′ region. Using the partial dog NPC1L1 sequence and the human sequence, genomic sequence for dog NPC1L1 was identified. Translation of an open reading frame extracted from the genomic sequence was in good agreement with human and bovine NPC1L1. Therefore, the primers dNL1-s (CTGCACAGGGATGGCGGACACTGGCCTGAG; SEQ ID NO: 2) and dNL1-s (CTCCGGCTTCATCAGAGGTCCGGTCCACTGC, SEQ ID NO: 3) were designed to amplify a product of approximately 4 Kbp using Phusion DNA polymerase in a high fidelity PCR reaction performed with single stranded cDNA and an extension time of 135 seconds and 33 cycles. PCR products from several reactions were combined and purified prior to cloning into the vector pcDNA5/FRT TOPO. Sequencing of several plasmids containing insert revealed a PCR product for the complete coding region of dog NPC1L1, with start and putative stop codons. Since the insert consistently integrated into pcDNA5/FRT TOPO in the reverse orientation, it was isolated by restriction digest, and directionally cloned into the vector pcDNA5/FRT.

MDCKII-Flp cells were generated by stably transfecting with pFRT/lacZeo cDNA (Invitrogen) using Lipofectamine 2000 (Invitrogen) according to manufacturer's instructions. Forty eight hours after transfection, cells were selected in zeocin (700 μg/ml), and resulting cell colonies were isolated and assayed for β-galactosidase activity (β-galactosidase assay kit, Invitrogen). The clone with the highest activity was used as the host cell line in subsequent transfections. Dog and human NPC1L1/MDCK II-Flp stable cell lines were generated by transfecting MDCKII-Flp cells with pcDNA5/FRTdog NPC1L1 or pcDNA5/FRT-human NPC1L1 plasmids using lipofectamine, followed by selection on 200 μg/ml hygromycin B. Clones were isolated with cloning rings and selected for levels of [³H]AS binding in the absence, or presence, of 10 mM sodium butyrate, in order to identify cells expressing high amounts of human or dog NPC1L1.

### Example 7

### Cloning and Pharmacological Characterization of The NPC1L1-Like Activity from MDCK II Cells

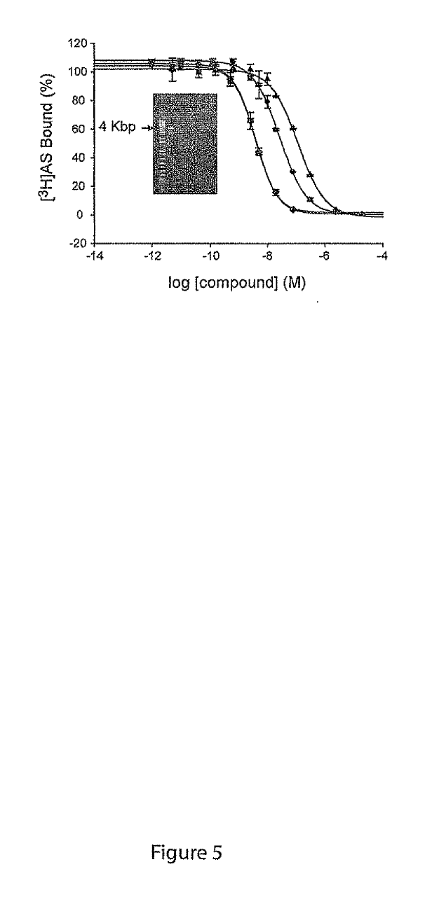

Given that [³H]AS binding data strongly suggest the presence of NPC1L1 in the apical membrane of MDCKII cells, total RNA was isolated from MDCKII cells in order to clone dog NPC1L1 cDNA (FIG. 5, inset) The isolated full length clone contains a single amino acid change from the predicted genomic sequence (1864M), and is in agreement with another recently reported dog NPC1L1 sequence; Hawes et al., 2007 Mol. Pharmacol. 71:19-29. Furthermore, our clone contains a single amino acid change from the recently reported dog NPC1L1 clone (L64P), in agreement with the predicted genomic sequence. Dog NPC1L1, like its homologues in other species is predicted to have 13 transmembrane domains, with N-terminus outside and C-terminus inside. Similarly, the sterol sensing domain (SSD) is conserved with that found in other species. These data strongly suggest that the NPC1L1-like activity from MDCK cells indeed represent dog NPC1L1 and is consistent with all the features of [³H]AS interaction with these cells (see below).

To further validate this statement, cloned dog NPC1L1 was transiently expressed in TsA201 cells and binding of [³]AS to these cells was then characterized (FIG. 5 and Table 1). Under equilibrium binding conditions, [³H]AS binds with a Kd of 2.15±0.39 nM and a Bₘₐₓ of approximately 5.68×10⁶ sites/cell (Table I). AS, PS, EZE-glue, and EZE inhibit [³H]AS binding to transiently transfected TsA201 cells with Kᵢ values of 1.00±0.11, 0.97±0.08, 5.51±1.52, and 21.48±7.56 nM, respectively. It appears that the differences in absolute K_{d} and Kᵢ values between MDCKII and dog NPC1L1-transfected TsA201 cells are the result of the transient over-expression in TsA201 cells. When over-expression is limited so that the Bₘₐₓ becomes equivalent to that of MDCKII cells, Kᵢ and K_{d} values become similar (data not shown), however, it is difficult to control for reduced levels of expression in transiently transfected TsA201 cells. Nonetheless, our data are consistent with dog NPC1L1 being endogenously expressed in MDCK cells.

### Example 8

### Surface Expression of NPC1L1 in MDCK Cells is Sensitive to Cell Cholesterol Levels

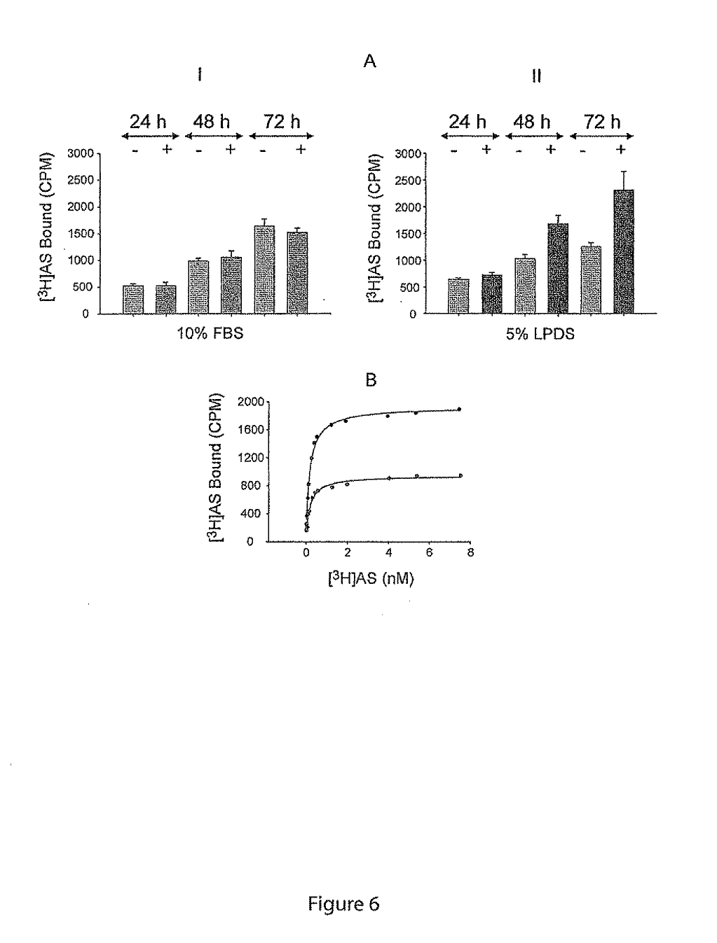

To determine whether the expression pattern of NPC1L1 in MDCKII cells is sensitive to changes in the endogenous concentration of cholesterol, MDCKII cells were seeded and grown in either 10% FBS or 5% lipoprotein deficient serum (5% LPDS) in the absence or presence of the HMG CoA reductase inhibitor, lovastatin. MDCKII cells grown in either 10% FBS or 5% LPDS, display an increase in the amount of [³H]AS binding from 24 to up to 72 h (FIG. 6A). Incubation of MDCKII cells with 4 μM lovastatin, does not cause any significant effect on the surface expression of NPC1L1 grown in 10% FBS (FIG. 6A, I). However, lovastatin treatment doubles [³H]AS binding in cells grown in 5% LPDS at 72 h (FIG. 6A, II). The increase in [³H]AS binding caused by lovastatin/5% LPDS is not due to enhanced [³H]AS affinity, K_{d} values of 180 in either case, but to an increase in the number of NPC1L1 sites at the cell surface, Bₘₐₓ of 75 pM (5% LPDS) and 154 pM (5% LPDS and 4 μM lovastatin), (FIG. 6B).

### Example 9

### Cell Based [3H]cholesterol OR [3H]Sitosterol Flux

Flux assays were performed essentially as described by Yu et al., 2006 J Biol. Chem. 281:6616-6624. Briefly, cell growth medium was completely aspirated and replaced with 200 μl of 5% LPDS containing the appropriate concentration of compound and incubated at 37° C./3 h in a 5% CO₂ incubator. Media was subsequently aspirated from cells and cells were incubated in 200 μl of 0-5.5% βmCD dissolved and filtered through a 0.22 μM filter at 37° C./45 minutes in a 5% CO₂ incubator. Media was dumped from cells that were then washed twice with 125 μl of 5% LPDS before media was aspirated and [3H]cholesterol complexed to BSA in 5% LPDS was added; see Yu et al., 2006 J. Biol. Chem. 281:6616-6624. After 45 minute incubation, cells were washed twice with DMEM, thoroughly aspirated and then 1% SDS was added prior to extraction for scintillation counting.

### Example 10

### Over-Expression of NPC1L1 in MDCKII-FLP Cells is Necessary for EZE-Like Sensitive [³H]Cholesterol Flux

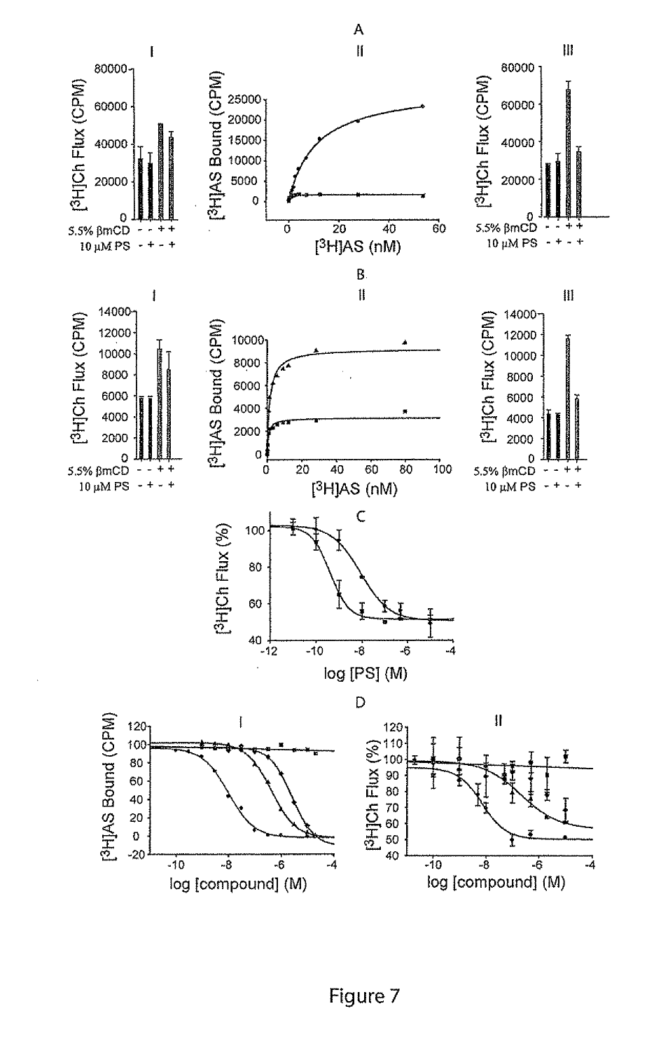

To validate MDCKII cells as an appropriate surrogate system for monitoring NPC1L1-dependent processes, we evaluated their ability to perform EZE-sensitive cholesterol flux using a similar protocol to that recently reported. This assay makes use of the ability of βmCD to deplete membrane-bound cholesterol. Subsequent exposure of cells to [³H] cholesterol provides a time-dependent flux of this substrate into the cells. However, pre-treatment of MDCKII-Flp cells with 5.5% βmCD only caused a small increase in [³H] cholesterol influx into the cells that was marginally blocked with 10 μM PS (FIG. 7A, I). In an attempt to improve the assay window, a stable MDCKII-Flp cell line over-expressing human NPC1L1, hNPC1L1/MDCKII-Flp, was generated. [³H]AS binding to MDCKII-Flp or hNPC1L1/MDCKII-Flp cells indicated that the expression of human NPC1L1 led to a change in K_{d} from 0.4 nM to 11 nM, as a consequence of the dramatic increase in levels of hNPC1L1, Bₘₐₓ increased from 73 pM (3.55×10⁵ sites/cell) in MDCKII-Flp cells to 1260 pM (6.07×10⁶ sites/cell) in hNPC1L1/MDCKII-Flp cells (FIG. 7A, II). Remarkably, in hNPC1L1/MDCKII-Flp cells, treatment with 5.5% βmCD led to a significant increase in the amount of [³H] cholesterol influx into cells that is almost completely blocked in the presence of 10 μM PS (FIG. 7A, III).

Further evidence for the role of NPC1L1 expression levels on EZE-sensitive [³H]cholesterol influx was obtained by analyzing the properties of MDCKII-Flp cells over-expressing dog NPC1L1 (dNPC1L1/MDCKII-Flp cells) in an inducible manner. Without induction, dNPC1L1/MDCKII-Flp cells bind [³H]AS with a K_{d} of 0.78 nM, and a Bₘₐₓ of 131 pM (6.23×10⁵ sites/cell, FIG. 7B, I). Following induction of dNPC1L1/MDCKII-Flp cells for 24 h with 4 mM sodium butyrate (Chen et al., 1997 Proc. Natl. Acad. Sci. USA 94:5798-5803, K_{d} remains similar at 1.53 nM, however, the Bₘₐₓ rises to 384 pM (1.83×106 sites/cell, FIG. 7B, II). Notably, after NPC1L1 induction, treatment of the cells with 5.5% βmCD leads to a significant increase in the amount of [³H] cholesterol entering cells and this process is almost completely blocked by 10 μM PS (FIG. 7B, III).

To further characterize the [³H] cholesterol influx process into MDCK cells, the potency of EZE-like compound PS for inhibiting [³H] cholesterol uptake was determined. [³H] cholesterol influx into both dNPC1L1/MDCKII-Flp and human NPC1L1/MDCKII-Flp cells was found to be sensitive to the presence of increasing concentrations of PS. IC₅₀ values for inhibition of [³H] cholesterol uptake, 0.32±0.09 and 10.3±1.5 nM for dNPC1L1/MDCKII-Flp and hNPC1L1/MDCKII-Flp, respectively, correlated well with corresponding K_{d} values, 0.8 and 11 nM, respectively (FIG. 7C). Furthermore, the rank order of potency of for a series of β-lactams as inhibitors of [³H]AS binding [(FIG. 7D, I), PS (5 nM)>>EZE-gluc (209 nM)>EZE (1.3 μM)>ent-1 (N.D., >100 μM)] correlates well with the IC₅₀ values of these compounds to block [³H] cholesterol influx [(FIG. 7D, II), PS (7 nM)>>EZE-Glut (300 nM)>EZE (N.D.>1 μM)>ent-[(N.D., >100 μM)]. In addition, [³H]sitosterol behaves in a similar manner to [3H]cholesterol in both dNPC1L1/MDCKII-Flp and hNPC1L1/MDCKII-Flp cells, in agreement with a previous report (Yamanashi et al., 2007 J. Pharmacol. Exp. Ther. 320(2):559-564) and in vivo pharmacology. These data, taken together, strongly support the notion that MDCKH cells represent a powerful functional system for studying NPC1L1-dependent processes.

1. A method for identifying an NPC1L1 modulator, which comprises:
(a) contacting MDCK cells or membrane preparation thereof with a candidate NPC1L1 modulator; and
(b) determining whether the candidate NPC1L1 modulator specifically binds to NPC1L1;
specific binding to NPC1L1 indicating an NPC1L1 modulator.

2. The method of claim 1 which further comprises:
(a) contacting MDCK cells or membrane preparation thereof with a detectably labeled known NPC1L1 modulator; and
(b) measuring the amount of bound detectably labeled known NPC1L1 modulator;
wherein a reduced amount of bound detectably labeled known NPC1L1 modulator in the presence of the candidate NPC1L1 modulator as compared to that measured in its absence indicates the presence of an NPC1L1 modulator.

3. The method of claim 2
wherein the known NPC1L1 modulator is selected from the group consisting of: substituted azetidinones, substituted 2-azetidinones, substituted 2-azetidinone-glucuronide, and ezetimibe-glucuronide.

4. (canceled)

5. The method of claim 3 wherein the known NPC1L1 modulator is selected from the group consisting of:
(a) EZE-gluc-enantiomer (“ent-1”);
(b) 4-[(2S,3R)-3-[(3S)-3-(4-fluorophenyl)-3-hydroxypropyl]-1-(4-{3-[(methylsulfonyl)amino]prop-1-yn-1-yl}phenyl)-4-oxoazetidin-2-yl]phenyl methyl-β-D-glucopyranosiduronate (“PS”); and
(c) alkyl sulphonamide, 4-[(2S,3R)-3-[(3S)-3-(4-fluorophenyl)-3-hydroxypropyl]-1-(4-{3-[(methylsulfonyl)amino]propyl}phenyl)-4-oxoazetidin-2-yl]phenyl β-D-glucopyranosiduronic acid (“AS”).

6. The method of claim 2 which comprises:
(a) saturating NPC1L1 binding sites on MDCK cells or membrane preparation thereof with a detectably labeled known NPC1L1 modulator;
(b) measuring the amount of bound detectably labeled known NPC1L1 modulator;
(c) contacting the cells or membrane preparation with an unlabeled or differently labeled candidate NPC1L1 modulator; and
(d) determining the amount of bound detectably labeled known NPC1L1 modulator remaining from (b);
wherein a reduced amount of bound detectably labeled known NPC1L1 modulator as compared to that measured in its absence indicates the presence of an NPC1L1 modulator.

7. (canceled)

8. (canceled)

9. The method of claim 2 which comprises:
(a) incubating MDCK cells or membrane fraction thereof with scintillation proximity assay (“SPA”) beads;
(b) contacting the SPA beads obtained from step (a) with:
(i) detectably labeled known NPC1L1 modulator and
(ii) a candidate NPC1L1 modulator; and
(c) measuring fluorescence to determine scintillation;
wherein a reduction of fluorescence as compared to that measured in the absence of the candidate NPC1L1 modulator indicates an NPC1L1 modulator.

10. (canceled)

11. (canceled)

12. A method for identifying an NPC1L1 modulator which comprises:
(a) incubating MDCK cells or membrane fraction thereof with SPA beads;
(b) contacting the SPA beads obtained from step (a) with detectably labeled candidate NPC1L1 modulator; and
(c) measuring fluorescence;
wherein detection of fluorescence indicates an NPC1L1 modulator.

13. (canceled)

14. The method of claim 2 which comprises:
(a) providing a plurality of fluorescer-bearing support particles bound to MDCK cells or membrane fraction thereof;
(b) contacting the particles with a radiolabeled known NPC1L1 modulator;
(c) contacting the particles with a candidate NPC1L1 modulator; and
(d) measuring emitted radioactive energy;
wherein a reduction in energy emission as compared to that measured in the absence of the candidate NPC1L1 modulator indicates an NPC1L1 modulator.

15. (canceled)

16. The method of claim 2 which comprises:
(a) providing, in an aqueous suspension, a plurality of fluorescer-bearing support particles attached to MDCK cells or membrane fraction thereof;
(b) contacting the suspension with a radiolabeled known NPC1L1 modulator;
(c) contacting the suspension with a candidate NPC1L1 modulator; and
(d) measuring emitted radioactive energy;
wherein a reduction in energy emission as compared to that measured in the absence of the candidate NPC1L1 modulator indicates an NPC1L1 modulator.

17. (canceled)

18. A method for identifying an NPC1L1 modulator which comprises:
(a) providing MDCK cells over-expressing NPC1L1;
(b) reducing or depleting cholesterol from plasma membrane of the cells;
(c) contacting MDCK cells with detectably labeled sterol or 5α-stanol and a candidate NPC1L1 modulator; and
(d) monitoring for an effect on cholesterol influx;
wherein a decrease in sterol or 5α-stanol influx as compared to that effected in the absence of the candidate NPC1L1 modulator indicates an NPC1L1 antagonist; and
wherein an increase of sterol or 5α-stanol influx as compared to that effected in the absence of the candidate NPC1L1 modulator indicating an NPC1L1 agonist.

19. (canceled)

20. (canceled)

21. The method of claim 18 where step (b) is carried out by the addition of methyl-β-cyclodextrin (“MβCD”).

22. (canceled)

23. (canceled)

24. The method of claim 18 which further comprises preparing a cell lysate from the MDCK cells between steps (c) and (d).

25. The method of claim 18 wherein the influx of detectably labeled sterol or 5α-stanol is measured by liquid scintillation counting.

26. The method of claim 18 wherein step (b) comprises
inhibiting or blocking endogenous cholesterol synthesis.

27. The method of claim 26 where step (b) is carried out by the addition of a statin.

28. (canceled)

29. (canceled)

30. (canceled)

31. (canceled)

32. Isolated NPC1L1 polypeptide which comprises SEQ ID NO: 5.

33. Isolated nucleic acid which comprises a sequence of nucleotides encoding SEQ ID NO: 5.

34. The isolated nucleic acid of claim 33 which comprises SEQ ID NO: 4.

35. A vector comprising the nucleic acid of claim 33.

36. A vector comprising the nucleic acid of claim 34.

37. An isolated population of MDCK cells expressing recombinant NPC1L1 protein or a membrane fraction thereof.


SEQUENCE LISTING (5 sequences)

SEQ ID NO: 1 - protein, 4 aa, Artificial Sequence
  YQRL Protein Motif in NPC1L1
YQRL

SEQ ID NO: 2 - DNA, 30 bp, Artificial Sequence
  PCR PRIMER
ctgcacagggatggcggacactggcctgag

SEQ ID NO: 3 - DNA, 31 bp, Artificial Sequence
  PCR PRIMER
ctccggcttcatcagaggtccggtccactgc

SEQ ID NO: 4 - DNA, 3978 bp, Canis familiaris
atggcggacactggcctgaggggctggctgctatgggcactgctcctgcatgtggcccagagtgagctgtacacacccatccaccagcctggctactgcgctttctacgacgagtgtgggaagaacccagagctgtctgggggactggcgcctctgtctaatgtgtcctgcctgtccaacacgcccgccccccgtgtcactggtgagcacctgaccctcctacagcgcatctgcccccgcctctacacgggcaccaccacctatgcctgctgctcccccaagcagctgctgtccctggagacgagcctggcggtcaccaaggccctcctcacccgctgccccacctgctccgacaactttgtgaacctgcactgccaaaacacctgcagccccaaccaaagtctcttcatcaacgtgacccgcgtggctgggggcgggggtggccggccccaggctgtggtggcctatgaggccttctaccaggacacctttgcccagcagacctacgactcttgcagccgggtgcgcatccctgcggctgccacgctggccgtgggcaccatgtgtggcgtttatggctccaccctctgcaatgctcagcgctggctcaatttccagggggacacttcgaatggcctggctcccctagacatcaccttccacctgatggagcccggccaggccctagggagtgggatgcaggctctgaccggggagatcaggccctgcaacgagtcccagggcaatggcacggtggcctgctcctgccaggactgtgctgcgtcctgccccaccatcccccagccccaggcactggactccaccttctacctgggcgggctggaaggtgggctggcccttgtcatcatcctctgctctgcttttgccctgcttaccaccttcctggtgggtacccgcctggcctcctcctgtggcaaggacaagacgccagaccccaaggcaggcatgagcctgtctgacaaactcagcctctccaccaacgtcatccttagccagtgcttccagaactggggcacatgggtggcctcatggccgctgaccatcctgttggtgtccatcgccgtggtattggccttgtcaggaggcctggcctttgtggaactgaccacggacccagtggagctgtggtcggcccccagcagccaagcccggagcgagaaggctttccacgaccagcattttggccccttcctccgaaccaaccaggtgatcttgacggctcccaaccggcccagctaccactacgactccctgctcctggggcccaagaacttcagtggggtcctggcctctgacctcctgctggagctgctggagctacaggagacgctgcggcacctccaggtgtggtcgcccgaggagcagcgccacatctcgctgcaggacatctgcttcgcgcccctcaaccctcacaatgccagcctctccgactgctgcatcaacagcctcctgcagtatttccagagcaaccgcacgcacctgctgctcacggccaaccagacgctgacgggccagacctcccaggtggactggagggaccactttctctactgtgctaacgccccactcaccttcaaggatggcacagccctagccctgagctgcatggctgactatgggggccctgtcttccccttccttgccgtgggtggctacaaagggaaggactactctgaggcggaggccctgattatgaccttctccctcaacaactatgcccctggggacccccggctggcccaggctaagctctgggaggcagccttcttggaggagatgaaagccttccagcggcggacagctggcactttccaggtcacattcatggctgagcgctccctggaggacgagattaaccgcacgacggcggaggacctccccatcttcggagtcagctacatcatcatcttcctgtacatctccctggcgctgggcagctactccagctggcgccgggtgccggtggactccaaggtcacgctgggcctgggcggggtggcggtggtgctgggagcagtgacagcggccatgggcttcttctcctacctcggcgtgccgtcctccctggtgatccttcaggtggtgcctttcctggtgttggccgtgggcgctgacaacatcttcatctttgttctggagtaccagaggctgccccggaggccgggagagccgcgggaggcccacatcggccgagcgctgggcagtgtggcccctagcatgttgctctgcagcctgtctgaggccatctgcttctttctaggggccctgacccctatgcccgctgtgaagacctttgccctgatctcgggctttgccatcgtcctggacttcttgctgcaggtgtcagcctttgtggctctgctttctctggacagcaggaggcaggaggcctcccgcttggacgtctgctgctgcgtgagcgccccgaagctgcctgcacccggccagagcgagggactcctgcttcgagtcttccgcaagttctacgtcccagtcctgctgcaccgggtgacacgggcggtggtgctgctgctgttcaccggcctcttcggggtggggctctacttcatgtgccacatccgcgtgggattggatcaggagctggccctgcccaaggactcatacctgctggactatttcttcttcctgaaccgctactttgaggtgggggctcccgtctactttgtcaccacgggaggctacaacttctccagcgaggcgggcatgaatgctgtgtgctccagtgccgggtgcgacagttactccttaacccagaagatccagtacgccaccgagttccccgaggagtcttacctggccatccctgcctcctcctgggtggatgacttcatcgactggctgaccccgtcctcctgctgccgcctttatgcctttggtgctaataaggacaaattctgcccttcgactgtcaactccctagcctgcttgaagaactgcgtgaacttcacactgggccctgtccggccatccgtggaccagttccacaagtaccttccctggttcctgagtgacccgcccaacatcaagtgtcccaaaggtgggctggcagcgtacaacacctccgtgcatttgggatctgatggccaggttttagcctcccggttcatggcctaccacaagccgctgcggaactcggaggattacactgaggccctgcgggtgtcacgggcgctggcggccaacatcacggcccagctgcggcaggtgccaggcaccgacccggccttcgaggtcttcccctacacgatcaccaacgtgttctacgagcagtacctgagcgtggtccccgagggcctcttcatgctcgccatctgcctgctgcccaccttcgtagtctgctgcctgctgctgggcatggacctacgctccggcctcctcaacctgttctccatcgtcatgatcctcgtggacaccgtgggcttcatggccctgtggggcatcagttacaatgccgtgtcgctcatcaacctggtcacggcggtgggcatctccgtggagtttgtgtcccacatcacccgctcctttgcagtcagcacccggcccacccggctggagagggccaaggaggccaccatctccatgggcagcgcggtgtttgctggcgtggccatgaccaacctgccgggcatcctcgtcctgggcctggccaaggcgcagctcatccagatcttcttcttccgcctcaacctcctcatcaccgtgctgggtctgctgcatggcctggtcttcctgccagtggtcctcagctacctcgggcctgatatcaatgcagctctcgtgctggaccagaagaagacagaagaggccatcggggcccctgcccacctggtcccaacatccacggccagcagcacctatgtcaactacggcttccaacatcccgccaacggtgtagtgggcgacagttctctgccccgcagtggaccggacctctga

SEQ ID NO: 5 - protein, 1325 aa, Canis familiaris
MADTGLRGWLLWALLLHVAQSELYTPIHQPGYCAFYDECGKNPELSGGLAPLSNVSCLSNTPAPRVTGEHLTLLQRICPRLYTGTTTYACCSPKQLLSLETSLAVTKALLTRCPTCSDNFVNLHCQNTCSPNQSLFINVTRVAGGGGGRPQAVVAYEAFYQDTFAQQTYDSCSRVRIPAAATLAVGTMCGVYGSTLCNAQRWLNFQGDTSNGLAPLDITFHLMEPGQALGSGMQALTGEIRPCNESQGNGTVACSCQDCAASCPTIPQPQALDSTFYLGGLEGGLALVIILCSAFALLTTFLVGTRLASSCGKDKTPDPKAGMSLSDKLSLSTNVILSQCFQNWGTWVASWPLTILLVSIAVVLALSGGLAFVELTTDPVELWSAPSSQARSEKAFHDQHFGPFLRTNQVILTAPNRPSYHYDSLLLGPKNFSGVLASDLLLELLELQETLRHLQVWSPEEQRHISLQDICFAPLNPHNASLSDCCINSLLQYFQSNRTHLLLTANQTLTGQTSQVDWRDHFLYCANAPLTFKDGTALALSCMADYGGPVFPFLAVGGYKGKDYSEAEALIMTFSLNNYAPGDPRLAQAKLWEAAFLEEMKAFQRRTAGTFQVTFMAERSLEDEINRTTAEDLPIFGVSYIIIFLYISLALGSYSSWRRVPVDSKVTLGLGGVAVVLGAVTAAMGFFSYLGVPSSLVILQVVPFLVLAVGADNIFIFVLEYQRLPRRPGEPREAHIGRALGSVAPSMLLCSLSEAICFFLGALTPMPAVKTFALISGFAIVLDFLLQVSAFVALLSLDSRRQEASRLDVCCCVSAPKLPAPGQSEGLLLRVFRKFYVPVLLHRVTRAVVLLLFTGLFGVGLYFMCHIRVGLDQELALPKDSYLLDYFFFLNRYFEVGAPVYFVTTGGYNFSSEAGMNAVCSSAGCDSYSLTQKIQYATEFPEESYLAIPASSWVDDFIDWLTPSSCCRLYAFGANKDKFCPSTVNSLACLKNCVNFTLGPVRPSVDQFHKYLPWFLSDPPNIKCPKGGLAAYNTSVHLGSDGQVLASRFMAYHKPLRNSEDYTEALRVSRALAANITAQLRQVPGTDPAFEVFPYTITNVFYEQYLSVVPEGLFMLAICLLPTFVVCCLLLGMDLRSGLLNLFSIVMILVDTVGFMALWGISYNAVSLINLVTAVGISVEFVSHITRSFAVSTRPTRLERAKEATISMGSAVFAGVAMTNLPGILVLGLAKAQLIQIFFFRLNLLITVLGLLHGLVFLPVVLSYLGPDINAALVLDQKKTEEAIGAPAHLVPTSTASSTYVNYGFQHPANGVVGDSSLPRSGPDL